(19) United States
(12) Patent Application Publication
(10) Pub. No.: US 2006/0026697 A1
(43) Pub. Date: Feb. 2, 2006

(54) Genetic immunization of transgenic animals with non-eukaryotic expression systems

(75) Inventors: Bill Hearl, Damascus, MD (US); Jill Ray, Rockville, MD (US); Teri Heiland, New Market, MD (US); David Grass, Princeton, NJ (US)

Correspondence Address: VINSON & ELKINS, L.L.P., 1001 FANNIN STREET, 2300 FIRST CITY TOWER, HOUSTON, TX, 77002-6760, US

(73) Assignee: Capital Genomix

(21) Appl. No.: 11/132,165

(22) Filed: May 18, 2005

Related U.S. Application Data
(60) Provisional application No. 60/591,435, filed on Jul. 27, 2004.

(51) Int. Cl. A01K 67/027

(52) U.S. Cl. 800/6; 800/18

(57) ABSTRACT

The present disclosure is directed to novel methods for genetic immunization which utilize a eukaryote host with a non-eukaryotic expression system. Preferably the non-eukaryotic expression system is a bacteriophage RNA Polymerase, for example T7 RNA Polymerase, which is preferably expressed in specific tissue(s) of the host. Genetic immunization occurs when the eukaryote host is exposed to a polynucleotide construct encoding a specific immunogen operably linked to a promoter recognized by the non-eukaryotic expression system, which generates expression of the immunogen, thereby eliciting a humoral and/or cellular immune response in the host. The present disclosure is also directed to transgenic animals that express the non-eukaryotic expression system.

### CROSS REFERENCE

This application claims priority to provisional patent application 60/591,435, filed Jul. 27, 2004.

### STATEMENT REGARDING FEDERALLY SPONSORED RESEARCH OR DEVELOPMENT

Not applicable.

### REFERENCE TO A “MICROFICHE APPENDIX”

Not applicable.

### BACKGROUND OF THE INVENTION

1. Field of the Invention

This disclosure generally relates to the field of immunology, and more specifically to methods of producing an immune response in transgenic animals with a non-eukaryotic expression system by introducing constructs into the animal model that encode immunogens under the control of the non-eukaryotic expression system.

2. Description of Related Art

Production and use of antibodies have become a standard part of disease research, treatment and prevention. Laboratory investigations into disease causes and mechanisms of action are heavily dependent on modern molecular biological techniques that depend on the ubiquitous use of antibodies, as are subsequently targeted drug design and treatment therapies. For example, identification of receptor-ligand pairs, characterization of protein interactions, treatment of diseases, and identification of potential drug compounds are routinely accomplished using antibodies. Antibodies can be used to deliver drugs or radiation to tumors, or as a therapy themselves, such as for treatment of particular breast cancers. Passive immunization also remains a prevalent preventive treatment for many diseases such as rabies, diphtheria, tetanus, botulism, snake-bite, hepatitis B, hepatitis A and measles, while recent studies show promise for treatment/prophylaxis of asthma with antibodies.

Although antibodies have many different uses, production of useful antibodies is limited by current methods. One notable obstacle is the production of sufficient numbers of the antibodies. Many immunogens, such as certain viruses, have not been adapted to grow to high titer in artificial culture conditions and therefore are not produced in sufficient quantities to elicit a robust immunogenic response. Although purified antigens can be isolated or produced by synthesizing peptides that represent the important immunologic domain of the surface antigen of a pathogen, these peptides frequently elicit a poor response without the use of adjuvant for enhancement of the immune response (Murphy et al., “Immunization Against Viruses,” in Virology, Fields et al., Eds., Raven Press, New York, pp. 349-370, 1985). Unfortunately, the most common adjuvant, Freund's adjuvant, cannot be used in humans and no comparable alternative exists.

Another problem with using synthetic peptides occurs when the antigenic portion of the antigen results from the juxtaposition of various protein domains due to the inherent folding of a protein. When this occurs, synthesis of an appropriate antigenic peptide may be impossible. Indeed, many times not only are the sequences of the domains involved in the antigenic site unknown, but also the particular domains themselves (Roit, Essential Immunology, Blackwell Scientific Publications, Sixth Ed., 1988). One technique for overcoming this problem is known as genetic immunization or nucleic acid immunization.

Genetic immunization involves introducing a nucleic acid sequence encoding an immunogen into the cells of an animal, which elicits an immune response and results in the production of antibodies (WO 90/11092). Here, the antigen is produced in vivo by the animal's cells, rather than generating and introducing the immunogen exogenously into the animal's cells. Genetic immunization is also capable of stimulating both cellular and humoral immune responses. This general concept has proved workable, an example being the production of antibodies to human growth hormone protein after mice were transformed by bombardment with microprojectiles coated with plasmids containing human growth hormone genomic sequences (Tang et al., (1992) Nature 356:152-54). The injection of plasmids encoding viral genes into muscle cells also appears to be efficacious in producing an antibody response (Davis et al., (1993) Hum. Mol. Genet. 2:1847-51).

Literature reports suggest that the magnitude of the antibody response is directly related to the amount of DNA injected and the number of doses given (Deck et al., (1997) Vaccine 15:71-78). Recent reports indicate, however, that bombarded or injected antigens are not guaranteed to produce antibodies (Iwasaki et al., (1999) Vaccine 17:2081-88). Therefore, while genetic immunization has many advantages, it routinely results in antibody responses that are low affinity and low titer. As a result, screening immunizations using anti-sera often provide unsatisfactory or uninformative results, and the researcher must turn to monoclonal antibodies to determine if the immunization was successful. In addition, genetic immunization has the risk that the nucleic acid sequence introduced will integrate into the recipient's genome, which may lead to long-term expression of a potentially harmful protein.

Liposomes have also been used to introduce polynucleotides into mammalian cells to provoke immunization, in some cases with as little as 5 μg of a DNA construct complexed with cationic lipid (WO 94/27435). These polycationic lipid complexes, as opposed to conventional liposomes that use anionic or neutral lipids, readily fuse with cell membranes, resulting in intracellular delivery of the polynucleotide. This type of delivery, which bypasses the lysosomal compartment and thus avoids degradation by lysosomal enzymes, is a major improvement over conventional techniques (Düzgünes et al., (1989) Biochemistry 28:9179-84; Felgner et al., (1989) Nature 337:387-88). Despite this, experimental results presented in WO 94/27435 indicate that immune responses to this system vary unpredictably, although antibodies were produced in all cases presented. Therefore, a need exists for a more reliable system to produce cellular and humoral immune responses to desirable immunogens.

### BRIEF SUMMARY OF THE INVENTION

The present disclosure addresses the shortcomings of the prior art with respect to producing cellular and humoral responses to specific immunogens by providing a eukaryote host, for example a transgenic non-human animal or transgenic vertebrate, genetically engineered to have enhanced receptivity or responsiveness to genetic immunization. This is achieved by utilizing a eukaryote host with a non-eukaryotic expression system that drives expression of a polynucleotide construct encoding an immunogen introduced into the host. In a preferred embodiment, the non-eukaryotic expression system includes a bacteriophage RNA polymerase under the control of a promoter that is recognized by the endogenous cellular machinery of the host. The bacteriophage RNA polymerase produced in the eukaryote host recognizes a promoter in an exogenously introduced construct that is operably linked to a polynucleotide encoding a specific immunogen. The bacteriophage RNA polymerase causes expression of the immunogen, thereby eliciting an immune response in the host to the immunogen. Preferably, expression of the bacteriophage RNA polymerase is tissue-specific or inducible in the eukaryote host.

Some advantages of the disclosed system are: (1) the polynucleotide encoding the immunogen does not need to enter the nucleus of a cell expressing the non-eukaryotic expression system to result in immunogen expression; (2) the polynucleotide does not create stable transfectants; (3) expression of the immunogen can be designed to be tissue-specific and transient; (4) expression of the non-eukaryotic expression system can be designed to be inducible; (5) in vitro cell lines which express various bacteriophage RNA polymerases are available for preparation of positive control extracts for anti-sera screening; and (6) the polynucleotide encoding the immunogen, which is under the control of a non-eukaryotic promoter, is not expressed in the eukaryotic host or tissues that lack the corresponding bacteriophage RNA polymerase.

Practical applications of the system disclosed herein are for generating antibodies or cytotoxic T-lymphocytes (CTLs) for use in laboratory research procedures, as clinical diagnostic reagents, as therapeutic antibodies for disease therapy, or for passive immunization. The system can also be used to generate monoclonal and polyclonal antibodies, as a source for humanized antibodies, and to prepare a human antibody library. In addition, the present disclosure is well-suited for developing antibodies to be used for treatment of cancer, tumors, autoimmunity, or allergy on an individualized basis, or panels of antibodies to be used in disease diagnosis. The instant disclosure also describes methods for generating eukaryote hosts that express a non-eukaryotic expression system for genetic immunization.

A preferred embodiment of the present disclosure is a transgenic non-human animal with a non-eukaryotic expression system, which is encoded by one or more transgenes incorporated into the genome of the animal. The transgenic animal is genetically engineered for greater responsiveness to genetic immunization. This embodiment includes all progeny of the transgenic animal which have the transgene incorporated into their genome. Preferably, the non-eukaryotic expression system includes a bacteriophage RNA polymerase, for example T7 or SP6 RNA Polymerase (U.S. Pat. No. 4,952,496; Butler and Chamberlin, (1982) J. Biol. Chem. 257:5772-78; each incorporated herein by reference). One preferred embodiment of a transgenic animal which expresses a bacteriophage RNA polymerase is a mouse model referred to herein as the “ImmunoMouse.” The ImmunoMouse preferably has a transgene incorporated into its genome that generates expression of T7 RNA polymerase in the mouse. Preferably the transgene includes a promoter operably linked to the T7 RNA polymerase that is tissue-specific and/or inducible. In a preferred embodiment, the tissue-specific promoter is a liver-specific, for example the ApoA1 promoter. In other preferred embodiments, the tissue-specific promoter is inducible.

After a eukaryote host with a non-eukaryotic expression system is generated, a polynucleotide construct is administered to the eukaryote host to elicit an immune response. As used herein, the term “polynucleotide construct” is interchangeable with the terms “recombinant vector,” “expression vector,” and other like terms well known to those of skill in the art. In a preferred embodiment, the polynucleotide construct encodes an immunogen under the control of a promoter recognized by the non-eukaryotic expression system in the eukaryote host. In preferred embodiments, the polynucleotide encoding the immunogen is operably linked to a promoter recognized by a bacteriophage RNA polymerase, for example T7, T3, or SP6 RNA Polymerase, and the appropriate bacteriophage RNA polymerase is expressed in the eukaryote host (U.S. Pat. Nos. 4,766,072 and 4,948,731, incorporated herein by reference). The bacteriophage RNA polymerase binds to the promoter of the polynucleotide construct, resulting in the in vivo expression of the immunogen. The immunogen elicits an immune response in the host, which preferably results in an antibody-based immune response or cell-mediated immune response to the immunogen. Antibodies or CTLs can then be isolated from the host, and used for a variety of applications, such as diagnostic and therapeutic applications.

In preferred embodiments, the polynucleotide construct is administered to the eukaryote host using a variety of routes, including but not limited to intramuscularly, intradermally, intravenously, intraperitoneally, or subcutaneously. In preferred embodiments, the polynucleotide construct is administered to the transgenic animal, for example an ImmunoMouse, through the tail vein or intramuscularly. The polynucleotide construct can be delivered to the eukaryote host as naked DNA, or for example, complexed with cationic lipid or another carrier. In one such preferred embodiment, the polynucleotide construct is delivered complexed with galactose-bearing polyethyleneimine (PEI-Gal) or other cationic polyethylenimine derivatives. The delivery of PEI-Gal-complexed polynucleotide constructs is particularly favored when the non-eukarotic expression system is under the control of a liver-specific promoter. This is because PEI-Gal has been shown to be highly efficient in the selective delivery of DNA constructs to hepatocytes. In other embodiments, the polynucleotide construct may include a tag sequence in frame with the immunogen that is useful for purification of the immunogen, or alternatively for monitoring immunogen production.

A preferred embodiment of the present disclosure is a eukaryotic host whose genome comprises a transgene comprising a DNA segment encoding a promoter operably linked to a bacteriophage RNA polymerase nucleic acid sequence, wherein the eukaryotic host exhibits enhanced responsiveness to genetic immunization as compared to control eukaryotic hosts. Another preferred embodiment of the present disclosure is the progeny of the eukaryotic host of the present disclosure, wherein the genome of the progeny comprises a transgene comprising a DNA segment encoding a promoter operably linked to a bacteriophage RNA polymerase nucleic acid sequence, wherein the progeny exhibit enhanced receptivity to genetic immunization as compared to control mice. In preferred embodiments, the eukaryotic host is a transgenic non-human animal, more preferably a transgenic mouse. The transgenic mice of the present disclosure may either be heterozygous, homozygous, or hemizygous for the transgene. In certain preferred embodiments, the transgenic mouse is a C57BL/6Bl hybrid strain. In preferred embodiments, the bacteriophage RNA polymerase encoded by the transgene is T7 RNA Polymerase or SP6 RNA Polymerase. In other embodiments, the promoter encoded by the transgene is tissue-specific, for example a liver-specific promoter such as ApoA1, or the promoter is inducible, for example a promoter that is induced by the eukaryotic host consuming alcohol.

Another preferred embodiment of the present disclosure is a method of generating antibodies against an immunogen, comprising introducing a polynucleotide construct comprising a nucleic acid sequence encoding an immunogen into a eukaryotic host, wherein the eukaryotic host expresses a bacteriophage RNA polymerase, wherein the bacteriophage RNA polymerase interacts with the polynucleotide construct to generate expression of the immunogen, wherein the immunogen elicits an immune response which results in the generation of antibodies. Preferably the eukaryotic host further comprises a transgene comprising a DNA segment encoding a promoter operably linked to a bacteriophage RNA polymerase nucleic acid sequence. In preferred embodiments, the eukaryotic host is a transgenic non-human animal, more preferably a transgenic mouse. In other preferred embodiments, the bacteriophage RNA polymerase encoded by the transgene is T7 RNA Polymerase or SP6 RNA Polymerase, and the promoter encoded by the transgene is tissue-specific, for example a liver-specific promoter, and/or an inducible promoter, for example a promoter that is induced by the eukaryotic host consuming alcohol. Examples of such promoters include Adh or ApoA1. Preferably the immunogen encoded by the polynucleotide construct is an antigen or an epitope. In other preferred embodiments, the polynucleotide construct further comprises a promoter specifically recognized by the bacteriophage RNA polymerase, wherein the bacteriophage RNA polymerase binds to the promoter and transcribes the nucleic acid sequence encoding the immunogen. In preferred embodiments, monoclonal antibodies against the immunogen are isolated from the eukaryotic host.

Yet another preferred embodiment of the present disclosure is a method of generating antibodies against a selected immunogen, comprising:

(a) introducing a polynucleotide construct comprising a nucleic acid sequence encoding an immunogen into a eukaryotic host, wherein the genome of the eukaryotic host comprises a transgene comprising a DNA segment encoding a promoter operably linked to a bacteriophage RNA polymerase nucleic acid sequence;

(b) inducing expression of the bacteriophage RNA polymerase in the eukaryotic host, wherein the bacteriophage RNA polymerase interacts with the polynucleotide construct to generate expression of the immunogen;

wherein the immunogen elicits an immune response which results in the generation of antibodies. In preferred embodiments, the eukaryotic host is a transgenic non-human animal, more preferably a transgenic mouse. Preferably the immunogen comprises an antigen or an epitope. In other preferred embodiments, the bacteriophage RNA polymerase encoded by the transgene is T7 RNA Polymerase or SP6 RNA Polymerase. In other embodiments, the promoter encoded by the transgene is tissue-specific, for example a liver-specific promoter such as ApoA1 or Adh, or inducible, for example a promoter that is induced by the eukaryotic host consuming alcohol. In certain embodiments, expression of the bacteriophage RNA polymerase is induced prior to introducing the polynucleotide construct into the eukaryotic host. In other preferred embodiments, the polynucleotide construct further comprises a promoter specifically recognized by T7 RNA Polymerase.

Another preferred embodiment of the present disclosure is a method of generating cytotoxic T-lymphocytes against an immunogen, comprising introducing a polynucleotide construct comprising a nucleic acid sequence encoding an immunogen into a eukaryotic host, wherein the eukaryotic host expresses a bacteriophage RNA polymerase, wherein the bacteriophage RNA polymerase interacts with the polynucleotide construct to generate expression of the immunogen, wherein the immunogen elicits an immune response which results in the generation of cytotoxic T-lymphocytes. Preferably the eukaryotic host further comprises a transgene comprising a DNA segment encoding a promoter operably linked to a bacteriophage RNA polymerase nucleic acid sequence. In preferred embodiments, the eukaryotic host is a transgenic non-human animal, more preferably a transgenic mouse. Preferably the immunogen comprises an antigen or an epitope. In other preferred embodiments, the bacteriophage RNA polymerase encoded by the transgene is T7 RNA Polymerase or SP6 RNA Polymerase. In other embodiments, the promoter encoded by the transgene is tissue-specific, for example a liver-specific promoter such as ApoA1 or Adh, or inducible, for example a promoter that is induced by the eukaryotic host consuming alcohol. In certain preferred embodiments, the polynucleotide construct further comprises a promoter specifically recognized by the bacteriophage RNA polymerase. In other preferred embodiments, the bacteriophage RNA polymerase binds to the promoter and transcribes the nucleic acid sequence encoding the immunogen.

Yet another embodiment of the present disclosure is a transgenic vertebrate whose genome comprises a bacteriophage RNA polymerase as a transgene, wherein the bacteriophage RNA polymerase transgene is capable of being expressed in at least one cell of the transgenic vertebrate. Preferably the vertebrate is a mammal, for example non-human primates, dogs, cats, sheep, pigs, goats, cattle, horses, ferrets, rats, rabbits, hamsters, gerbils, and mice. In certain embodiments, the bacteriophage RNA polymerase is T7, SP6, or T3 RNA polymerase. In other embodiments, the transgene is operably linked to a promoter, wherein the promoter is preferably a constitutive promoter or an inducible promoter.

Other embodiments of the present disclosure are methods of expressing a protein in a transgenic vertebrate whose genome comprises a bacteriophage RNA polymerase transgene, comprising: a) providing a construct comprising the following elements operably linked: i) a promoter sequence cognate to the bacteriophage RNA polymerase, ii) a eukaryotic ribosome recognition sequence, and iii) a sequence encoding the protein; b) introducing the construct into at least one cell of the transgenic vertebrate; and c) providing conditions whereby the transgenic vertebrate expresses said protein. Preferably the vertebrate is a mammal, for example non-human primates, dogs, cats, sheep, pigs, goats, cattle, horses, ferrets, rats, rabbits, hamsters, gerbils, and mice. In certain embodiments, the bacteriophage RNA polymerase is T7, SP6, or T3 RNA polymerase. In other embodiments, the transgene is operably linked to a promoter, wherein the promoter is preferably a constitutive promoter or an inducible promoter. In still other embodiments, the construct further comprises a stop codon, a tag sequence, a poly-adenosine tail or a combination of these elements. The methods disclosed above may also further comprise the step of isolating the protein.

Another embodiment of the present disclosure is a method to produce at least one antibody against an antigen in a transgenic vertebrate whose genome comprises a bacteriophage RNA polymerase transgene, comprising: a) providing a immunogenic construct comprising the following elements operably linked: i) a promoter sequence cognate to said bacteriophage RNA polymerase, ii) a eukaryotic ribosome recognition sequence, and iii) a sequence encoding said antigen; b) introducing said immunogenic construct into at least one cell of said transgenic vertebrate; and c) providing conditions whereby said transgenic vertebrate produces said at least one antibody against said antigen. Preferably the vertebrate is a mammal, for example non-human primates, dogs, cats, sheep, pigs, goats, cattle, horses, ferrets, rats, rabbits, hamsters, gerbils, and mice. In certain embodiments, the bacteriophage RNA polymerase is T7, SP6, or T3 RNA polymerase. In other embodiments, the transgene is operably linked to a promoter, wherein the promoter is preferably a constitutive promoter or an inducible promoter. In still other embodiments, the immunogenic construct further comprises a stop codon, a tag sequence, a poly-adenosine tail or a combination of these elements. The method disclosed above may also further comprise the step of isolating at least one antibody as a polyclonal antibody. Alternatively, the methods disclosed above may also further comprise the steps of collecting spleen cells from said transgenic vertebrate, making at least one hybridoma from said spleen cells, and isolating said at least one antibody as a monoclonal antibody from said at least one hybridoma.

Yet another embodiment of the present disclosure is a method to produce a transgenic vertebrate whose genome comprises a bacteriophage RNA polymerase transgene, comprising: a) introducing into the pronucleus of a fertilized ovum of a vertebrate a construct comprising a bacteriophage RNA polymerase as a transgene; b) transplanting said ovum into a female of said vertebrate; and c) allowing said ovum to develop to term, thereby producing a founder transgenic vertebrate individual. Preferably the vertebrate is a mammal, for example non-human primates, dogs, cats, sheep, pigs, goats, cattle, horses, ferrets, rats, rabbits, hamsters, gerbils, and mice. In certain embodiments, the bacteriophage RNA polymerase is T7, SP6, or T3 RNA polymerase. In other embodiments, the transgene is operably linked to a promoter, wherein the promoter is preferably a constitutive promoter or an inducible promoter. In still other embodiments, the method disclosed above further comprises the step of breeding the founder transgenic vertebrate individual to obtain F1 transgenic vertebrates homozygous or hemizygous for the transgene.

### BRIEF DESCRIPTION OF THE SEVERAL VIEWS OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 Illustration of plasmid ApoA1T7, from which a T7 RNA Polymerase transgene fragment was isolated and used to generate transgenic mice that express T7 RNA Polymerase in liver. A 2.7 kb fragment encoding T7 RNA Polymerase was functionally inserted downstream of the ApoA1 promoter (a liver-specific promoter) and upstream of the SV40 intron. The transgene fragment was released from plasmid ApoA1T7 by digesting the plasmid with SalI and XbaI.

FIG. 2 Southern blot analysis of founder mice genomic DNA probed with T7 RNA Polymerase sequence specific probes. Results indicate stable integration of the T7 RNA Polymerase transgene in two founder strains of mice, designated 15894 and 15916.

FIG. 3 Northern Blot analysis of RNA derived from the tissues of ImmunoMouse G1 progeny probed with a T7 RNA Polymerase sequence specific probe. Expression of T7 RNA Polymerase was only detected in liver of the ImmunoMouse, and not in any of the other tissues examined.

FIG. 4 ImmunoMouse animals generate antibodies against a known bacterial antigen, β-galactosidase. The results demonstrate that ImmunoMouse animals injected with a plasmid construct encoding β-galactosidase operably linked to a T7 RNA Polymerase promoter (pCGI-βgal) have an immune response that is approximately 70% of that observed with direct β-galactosidase protein immunization in Balb/C mice. ImmunoMouse animals injected with a plasmid construct lacking the β-galactosidase sequence (pCGI) had a poor antibody response, as did Balb/C mice injected with a plasmid construct encoding β-galactosidase operably linked to a CMV promoter (CMV-βgal), demonstrating the limitations of a traditional genetic immunization construct. Balb/C mice injected directly with β-galactosidase protein had the best immune response of the four groups.

FIG. 5 The titration curve of the four groups evaluated in FIG. 4 demonstrates that the decay of antibodies derived from the ImmunoMouse is very similar to the decay observed with protein immunization.

FIG. 6 ImmunoMouse animals showed a positive immune response to TIMP-2 after 21 days.

FIG. 7 Map of CGI-3941 plasmid constructs. Three different fragments of the CGI-3941 gene were cloned into a vector under control of the T7 promoter. The N-term fragment consists of 0.5 kb of DNA sequence from the 5′ end of the gene. The 2.3 kb fragment consists of the 3′ 2.3 kb of the translated piece of the gene. The C-term fragment consists of 0.3 kb of the 3′ fragment of the gene.

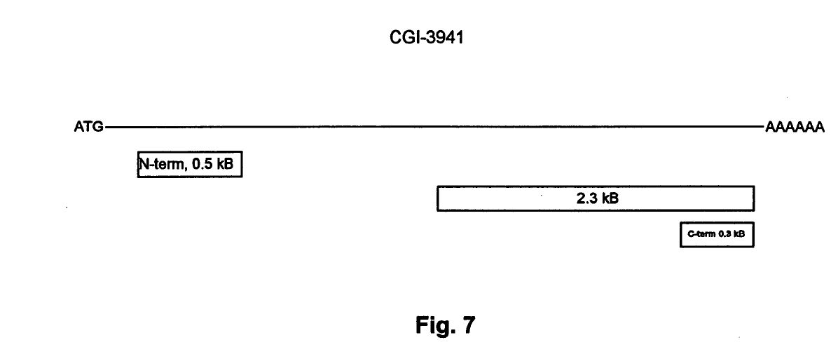

FIG. 8 ELISA assay average absorbance of D-56 anti-CGI-3941 sera at a serum dilution of 1:100.

### DETAILED DESCRIPTION OF THE INVENTION

The present disclosure is directed to a novel method for genetic immunization which utilizes a eukaryote host with a non-eukaryotic expression system. Preferably the non-eukaryotic expression system of the host includes a bacteriophage RNA polymerase under the control of a promoter that is recognized by the host's endogenous cellular machinery. Genetic immunization occurs when the eukaryote host is exposed to a polynucleotide construct encoding a specific immunogen operably linked to a promoter recognized by the non-eukaryotic expression system. The non-eukaryotic expression system generates expression of the immunogen, thereby eliciting an immune response in the host. Preferably, expression of the immunogen in the host is tissue-specific or inducible. As used herein, an “immunogen” is any agent or compound, including any antigen, that stimulates or causes an immune response when introduced into a host. Proteins or peptides that contain one or more epitopes are common immunogens. Preferably, the immunogen stimulates a humoral or cellular immune response that results in production of a specific antibody, antibodies, or cytotoxic T-lymphocytes (CTLs) that recognize the immunogen.

A. Eukaryote Host

A eukaryote host of the present disclosure is any eukaryote host that can be genetically engineered or otherwise manipulated to have a non-eukaryotic expression system. While the eukaryote host can be any eukaryotic organism, including but not limited to plants, transgenic non-human animals are preferred. Examples of suitable non-human animals include but are not limited to farm animals such as cattle, sheep, pigs, goats and horses; domestic mammals such as dogs and cats; laboratory animals such as rodents, mice, rats, rabbits, and guinea pigs; and birds, including domestic, wild, and game birds such as chickens, turkeys and other gallinaceous birds, ducks, geese, and the like. In a preferred embodiment, the transgenic non-human animal has a non-eukaryotic expression system. Preferably the non-eukaryotic expression system is encoded by one or more transgenes incorporated into the genome of the eukaryote host. The present disclosure encompasses not only eukaryotic organisms manipulated to have a non-eukaryotic expression system, but also all progeny of the organisms that maintain the non-eukaryotic expression system.

As used herein, the phrase “incorporated into its genome” is intended to refer to eukaryote hosts that have a selected transgene introduced into their germ cells and/or somatic cells such that the transgene is stably incorporated into the genome of the cell and is capable of carrying out a desired function. The term “genome” is intended to include the entire DNA complement of an organism, including the nuclear DNA component, chromosomal or extrachromosomal DNA, as well as the cytoplasmic domain (e.g., mitochondrial DNA). Thus, the present disclosure contemplates that one or more transgenes may be stably incorporated into the germ cells and/or somatic cells of an organism, in a functional form to achieve a desired effect, such as conferring a selected trait onto the transgenic animal.

B. Transgenes Encoding the Non-Eukaryotic Expression System

In preferred embodiments of the present disclosure, one or more transgenes encoding a non-eukaryotic expression system are transferred into a fertilized embryo or embryonic stem cells to produce a eukaryote host that expresses the non-eukaryotic expression system. As used herein, the term “transgene” is intended to refer broadly to any desired DNA sequence or nucleic acid construct that can be incorporated into a host's genome, including but not limited to genes or DNA sequences that are not normally present in the genome, genes or DNA sequences that are normally present, but are not normally transcribed and translated (“expressed”) in a given genome, or any other genes or DNA sequences that one desires to introduce into the genome. This may include genes or DNA sequences that are normally present in the non-transgenic genome but which one desires to alter the expression of, or which one desires to introduce in an altered or variant form. The nucleic acid sequence encoded by the transgene can include, but is not limited to, prokaryotic sequences, prokaryotic RNA, eukaryotic sequences, eukaryotic mRNA, cDNA from eukaryotic mRNA, genomic DNA sequences from eukaryotic DNA, and even synthetic DNA sequences.

The transgene of the present disclosure preferably encodes a bacteriophage RNA polymerase that is expressed in the eukaryote host. Examples of suitable bacteriophage RNA polymerases for the present disclosure include but are not limited to T7, T3, SP6, φ, φII, W31, H, Y, A1122, cro, C21, C22, C23, Pseudomonas putida phage gh-1, Serratia marcescens phage IV, Citrobacter phage ViIII, and Klebsiella phage number 11. Bacteriophage RNA polymerases are generally single polypeptide chains of less than 100 kDa. In preferred embodiments, the bacteriophage RNA polymerase expressed in the transgenic animals is T7 RNA Polymerase. The bacteriophage RNA polymerase may be encoded by genomic sequences, cDNA sequences, or fragments thereof, including but not limited to sequences that contain additions, deletions, substitutions, silent or conservative mutations or homologous sequences. The transgene also generally includes linked regulatory element(s) that serve to direct transcription and translation of bacteriophage RNA polymerase in the eukaryote host.

Homologous sequences include sequences that hybridize under moderate to high stringency conditions. The basic parameters affecting the choice of hybridization conditions and guidance for devising suitable conditions are set forth by Sambrook et al. 1989, Molecular Cloning: A Laboratory Manual 2^{nd} Ed. Cold Spring Harbor Laboratory Press, Cold Spring Harbor, N.Y., specifically incorporated herein by reference. As used herein, conditions of moderate stringency can be readily determined by those having ordinary skill in the art based on, for example, the length and/or base composition of the DNA. For hybridizing sequences longer than about 100 nucleotides with filter-bound target DNA or RNA, one way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 42° C. (or other similar hybridization solutions, such as one containing about 50% formamide, with a hybridization temperature of about 42° C.), and washing conditions of about 60° C., in 0.5×SSC, 0.1% SDS.

Conditions of high stringency can also be readily determined by the skilled artisan based on, for example, the length and base composition of the DNA. Generally, such conditions are defined as hybridization conditions as above, but with washing at approximately 68° C., 0.2×SSC, 0.1% SDS. It should be understood that the wash temperature and wash salt concentration can be adjusted as necessary to achieve a desired degree of stringency by applying the basic principles that govern hybridization reactions and duplex stability, as known to those skilled in the art (see, e.g., Sambrook et al., (1989)). It should be further understood that hybridization conditions for oligonucleotide probes of defined length and sequence can be designed by applying formulae known in the art (e.g., see Sambrook et al., (1989) at 11.45-11.47).

In another embodiment, the nucleic acid sequence encoding the bacteriophage RNA polymerase also comprise nucleotide sequences that are at least 80% identical to known bacteriophage RNA polymerase nucleic acid sequences. Also contemplated are embodiments in which a nucleic acid sequence comprises a sequence that is at least 90% identical, at least 95% identical, at least 98% identical, at least 99% identical, or at least 99.9% identical to known sequences. The percent identity may be determined by visual inspection and mathematical calculation. Alternatively, the percent identity of two nucleic acid sequences can be determined by comparing sequence information using the GAP computer program, version 6.0 described by Devereux et al., (1984) Nucl. Acids Res. 12:387, incorporated herein by reference) and available from the University of Wisconsin Genetics Computer Group (UWGCG). The preferred default parameters for the GAP program include: (1) a unary comparison matrix (containing a value of 1 for identities and 0 for non-identities) for nucleotides, and the weighted comparison matrix of Gribskov and Burgess (1986) Nucl. Acids Res. 14:6745, as described by Schwartz and Dayhoff, eds., Atlas of Protein Sequence and Structure, National Biomedical Research Foundation, pp. 353-358, 1979, each incorporated herein by reference; (2) a penalty of 3.0 for each gap and an additional 0.10 penalty for each symbol in each gap; and (3) no penalty for end gaps. Other programs used by one skilled in the art of sequence comparison may also be used.

The bacteriophage RNA polymerase is preferably operably linked to an expression control element, for example a heterologous promoter that is recognized by the host's cellular expression machinery. A heterologous sequence is one that is not operably linked or contiguous to another sequence in nature, and elements that are operably linked or contiguous to each other in nature may become heterologous if a filler sequence is placed between them. As used herein, “filler sequence” refers to any nucleotide sequence that is inserted into a polynucleotide construct to give spacing between particular components, such as a promoter and a coding region. The filler sequence may provide an additional attribute, for example one or more restriction enzyme recognition sites.

A “promoter” is a DNA sequence that directs transcription of a nucleic acid sequence. Typically a promoter is located in the 5′ region of a polynucleotide to be transcribed, proximal to the transcriptional start site of the nucleic acid sequence. The promoter used to drive expression of a bacteriophage RNA polymerase in a eukaryote host as disclosed herein is preferably of eukaryotic origin, but can also be a viral or retroviral promoter. In addition, the promoter operably linked to the bacteriophage RNA polymerase can be of any type recognized by the host, including but not limited to constitutive, inducible, cell-specific and/or tissue-specific promoters, all of which are well known to those of skill in the art. Strong promoters that produce high levels of mRNA transcripts are preferred. As used herein, the term “operably linked” means a linkage in which an expression control element such as a promoter sequence or promoter control element is connected to a nucleic acid sequence (or sequences) in such a way as to place transcription of the nucleic acid sequence under the influence or control of the expression control element. Preferably, two nucleic acid sequences that are operably linked do not result in the introduction of a frame-shift mutation in the transcribed sequence.

A constitutive promoter actively promotes transcription under most, but not necessarily all, environmental conditions and states of development or cell differentiation. An inducible promoter is a promoter which is regulated under certain conditions, such as light, chemical concentration, protein concentration, conditions in an organism, cell, or organelle, and the like. Examples of environmental conditions that may affect transcription by an inducible promoter include but are not limited to anaerobic conditions, elevated temperature, or the presence of light. An inducible promoter preferably has a high preference for being induced in a specific tissue or cell, and/or at a specific time during development of an organism. By “high preference” is meant at least about a 3-fold, 5-fold, 10-fold, 20-fold, 50-fold or 100-fold increase in transcription in the desired tissue over transcription in any other tissue. Preferably the promoter of the transgene is inducible through diet additives provided to the transgenic animal. A variety of inducible promoter systems have been described in the literature and can be used in the present disclosure, including, but not limited to, tetracycline-regulatable systems (WO 94/29442; WO 96/40892; and WO 96/01313, each incorporated herein by reference); hormone-responsive systems, interferon-inducible systems, metal-inducible systems, heat-inducible systems (WO 93/20218, each incorporated herein by reference); and ecdysone-inducible systems.

Promoters that may be used in the present disclosure comprise at least a basal promoter, which is the minimal sequence necessary for assembly of a transcription complex required for transcription initiation. Basal promoters frequently include a “TATA box” element, which is usually located between 15 and 35 nucleotides upstream from the site of initiation of transcription. Basal promoters also sometimes include a “CCAAT box” element (typically a sequence CCAAT) and/or a GGGCG sequence, usually located upstream from the start site of transcription. In preferred embodiments, the promoter used in the transgene is tissue-specific, which means that the promoter preferentially drives expression of the operably linked nucleic acid sequence in select tissues. Preferred tissue-specific promoters are promoters that result in expression in brain, skeletal muscle, heart, pancreas, liver, kidney, spleen, and the like, which are well known to those of skill in the art. More preferably the promoter used in the transgene is both tissue-specific and inducible. Examples of promoters and enhancers that may be optimal for use in the present disclosure are the promoter or enhancer regions for alcohol dehydrogenase (ADH); alcohol dehydrogenase 6 (ADH6); alpha 1-microglobulin/bikunin (ABP); cytomegalovirus (CMV); apolipoprotein E (Apo E); apolipoprotein C-I (Apo C-I); apolipoprotein AI (Apo AI); cholesterol 7 alpha-hydroxylase (CYP7); clotting factor IX (FIX); human alpha-antitrypsin (hAAT); and hepatic control region-1 (HCR-1). In other embodiments, these promoter regions may be used alone or alternatively linked to albumin or hepatitis B enhancers. (See Kramer et al., (2003) Mol Ther. 2003 7:375-85; Gehrke et al., (2003) Gene 322:137-43, each incorporate herein by reference).

In addition, the promoter may also include other regulatory sequences that influence transcription or translation initiation and rate, stability, and/or mobility of a transcript or polypeptide product. Preferably, regulatory sequences that confer high level expression and/or a cell-type-specific expression pattern, which are well known to those of skill in the art, are utilized for regulating expression of the transgene. Regulatory sequences include, but are not limited to, promoter control elements, protein binding sequences, 5′ and 3′ untranslated regions (UTRs), transcriptional start sites, termination sequences, polyadenylation sequences, introns, as well as certain sequences within amino acid coding sequences such as secretory signals, protease cleavage sites, and the like. A UTR is any contiguous series of nucleotide bases that are transcribed, but not translated. A 5′ UTR is located between the start site of the transcript and the translation initiation codon and includes the +1 nucleotide. A 3′ UTR is located between the translation termination codon and the end of the transcript. UTRs can have particular functions such as increasing mRNA stability or translation attenuation. Examples of 3′ UTRs include, but are not limited to, polyadenylation signals and transcription termination sequences.

Examples of promoter control elements include but are not limited to enhancers, scaffold attachment regions, Upstream Activating Regions (UARs), Upstream Repressor Regions (URRs), and other transcription factor binding sites and inverted repeats. An enhancer is a regulatory DNA sequence that is capable of activating transcription from a promoter linked to it. Enhancers can function in both orientations either upstream or downstream of a promoter. A scaffold attachment region is a nucleic acid sequence that anchors chromatin to the nuclear matrix or scaffold to generate loop domains that can have either a transcriptionally active or inactive structure (Spiker and Thompson (1996) Plant Physiol. 110:15-21; Whitelaw et al., (2000) Gene 244:73-80). A UAR is a position or orientation dependent nucleic acid element that primarily directs tissue, organ, cell type, or environmental regulation of transcript levels, usually by affecting the rate of transcription initiation. Corresponding DNA elements that have a transcription inhibitory effect are called URRs. Thus the term “promoter” as used herein can also refer to a region of sequence determinants located upstream of the start of transcription which are involved in recognition and binding of RNA polymerase and other proteins to initiate and modulate transcription.

The promoter is linked to the nucleic acid sequence encoding the bacteriophage RNA polymerase using standard molecular biology techniques that can be found in common laboratory manuals such as Molecular Cloning: A Laboratory Manual (Sambrook and Russell, 2001, Cold Spring Harbor Laboratory, Cold Spring Harbor, N.Y.) and Current Protocols of Molecular Biology (Ausubel et al., 2001, John Wiley & Sons, New York), each incorporated herein by reference. The resulting DNA sequence may then be inserted into a cloning vehicle such as a plasmid. Customized plasmid vectors can be constructed de novo or can be derived from commercially available expression vectors. For example, the pGL3-Basic Vector (Promega) and the pd2EGFP-N1 vector (Clontech) are promoterless vectors whose reporter genes can be excised after cleavage with Nco I and Xba I prior to insertion of the promoter-RNA polymerase polynucleotide. Alternatively, the bacteriophage RNA polymerase encoding nucleic acid sequence can be cloned directly into expression vectors already containing an appropriate promoter. Whether a customized vector or a commercially available vector is used, additional eukaryotic regulatory sequences discussed above can be included. The transgene can also include a selectable marker to facilitate the utility of the transgene.

Positive selection expression cassettes used in a transgene encode a selectable marker that affords a means for selecting cells that have integrated the transgene sequence spanning the positive selection expression cassette. Suitable drug resistance genes are well known to those of skill in the art and include but are not limited to: gpt (xanthine-guanine phosphoribosyltransferase), which can be selected for with mycophenolic acid; neo (neomycin phosphotransferase), which can be selected for with G418 or hygromycin; and DFHR (dihydrofolate reductase), which can be selected for with methotrexate (Mulligan and Berg, (1981) Proc. Natl. Acad. Sci. USA 78:2072; Southern and Berg, (1982) J. Mol. Appl. Genet. 1:327, each incorporated herein by reference). Positive selection involves expression from the expression cassette, which encodes a functional protein (e.g., neo or gpt) that confers a selectable phenotype to targeted cells harboring the endogenously integrated expression cassette, such that by addition of a selection agent (e.g., G418 or mycophenolic acid), targeted cells have a growth or survival advantage over cells that do not have an integrated expression cassette.

In a preferred embodiment of the present disclosure, the transgene present in a transgenic animal encodes T7 RNA Polymerase. An exemplary description of the elements of a transgene that may be utilized in the present disclosure follows, and the various embodiments of this transgene are applicable to transgenes encoding any bacteriophage RNA polymerase. The transgene for T7 RNA Polymerase has structural sequences encoding T7 RNA Polymerase, including but not limited to genomic sequences, cDNA sequences, and fragments thereof, including but not limited to sequences that contain additions, deletions, substitutions, silent or conservative mutations or homologous sequences. In general, regulatory element(s) are linked to the structural sequenced to drive expression of T7 RNA Polymerase in the transgenic animal. The transgene may include one or more introns or exons, and may encode a fusion protein which retains the ability to activate expression of a promoter recognized by T7 RNA Polymerase. Preferably, the linked regulatory element(s) are tissue-specific or inducible regulatory elements, rather than constitutive expression elements. The regulatory element(s) drive expression by providing transcriptional and translational initiation regions associated with gene expression, and functional transcriptional and translational termination regions. In other preferred embodiments, expression of the T7 RNA Polymerase in the transgenic animal is organ-specific, for example inducible expression in the liver.

Another strategy for driving expression of a bacteriophage RNA polymerase in a eukaryote host is to utilize endogenous regulatory elements in the genome of the particular host. For example, the transgene may be designed to integrate into a chromosomal location containing functional endogenous regulatory elements that are suitable for expression of the bacteriophage RNA polymerase encoded by the transgene. A transgene which utilizes endogenous regulatory elements of the host would preferably be introduced into a specific location of the host's genome using homologous recombination, as described in Thomas and Capecchi, (1987) Cell 51:503-12, and U.S. Pat. No. 5,464,764, incorporated herein by reference. One of skill in the art is familiar with the elements that must be present in a transgene construct for targeted homologous recombination. Endogenous regulatory elements include sequences that are natural to the host's genome, as well as sequences present in the host's genome as a result of an infectious disease, e.g. virus, prion, and the like.

Transgene constructs are typically cloned in bacterial cells and then isolated using standard molecular biology methods; alternatively, a transgene may be synthesized as oligonucleotides. Once transgenes are generated which encode the desired non-eukaryotic expression system, for example a transgene construct comprising a heterologous promoter operably linked to a nucleic acid sequence encoding a bacteriophage RNA polymerase, the transgene is introduced or transformed into a eukaryote host. The transgene can be introduced into the host cell using any one of a number of techniques well known to those of skill in the art. For example, host cells can be transfected using techniques described in common laboratory manuals such as Molecular Cloning: A Laboratory Manual (Sambrook and Russell, 2001, Cold Spring Harbor Laboratory, Cold Spring Harbor, N.Y.), and Current Protocols of Molecular Biology, each incorporated herein by reference. Suitable techniques include but are not limited to microinjection (Gordon et al., (1980) Proc. Natl. Acad. Sci. USA 77:7380-7384; Gordon & Ruddle, (1981) Science 214:1244-1246; U.S. Pat. No. 4,873,191), calcium-phosphate precipitation (Freshney, (1993) In: Culture of Animal Cells, third edition, Wiley-Liss, New York), DEAE-dextran, protoplast fusion, electroporation (Lo, (1983) Mol. Cell. Biol. 3:1803-1814), particle bombardment (Heiser, (1994) Annal. Biochem. 217:185-96), viral-based vectors, retrovirus mediated gene transfer (Van der Putten et al., (1985) Proc. Natl. Acad. Sci. USA 82: 6148-6152; Toyoshima and Vogt, (1969) Virology 38:414426; Coelen et al., (1983) Arch. Virol. 75:307-311; Chaney et al., (1986) Somatic Cell Mol. Genet. 12:237-244; Aubin et al., (1988) Somatic Cell Mol. Genet. 14:155-167, and Chisholm et al., (1988) Nuc. Acids Res. 16:2352), sperm-mediated gene transfer (Lavitrano et al., (1989) Cell 57:717-723); cationic lipid fusion (WO 94/27435), Polybrene, lipofection, chemical transfection, and homologous recombination (Thompson et al., (1989) Cell 56:313-321) (each reference incorporated herein by reference).

These methods can be used to generate either transiently transfected cells or stably transfected cells. The transgene can either insert randomly into the genome of the recipient cell, or recombine in a site-specific manner via homologous recombination. Preferably the transgene randomly inserts and stably incorporates into the genome of the eukaryote host. Stably transfected cells can also be obtained using enzyme-assisted site-specific integration systems that integrate a DNA molecule at a predetermined location. Examples, of such enzyme-assisted integration systems include the Cre/lox target system (e.g., as described in Baubonis and Sauer, (1993) Nuc. Acids. Res. 21:2025-2029; Fukushige and Sauer, (1992) Proc. Natl. Acad. Sci. USA 89:7905-7909; and U.S. Pat. No. 4,959,317), the FLP/FRT target system (e.g., as described in Dang and Pernimon, (1992) Dev. Genet. 13:367-375; Fiering et al., (1993) Proc. Natl. Acad. Sci. USA 90:8469-8473; and U.S. Pat. No. 5,654,182), the Gin recombinase of phage Mu, the Pin recombinase of E. coli, and the R/RS system of the pSRI plasmid (each reference incorporated herein by reference). The Cre/lox and FLP/FRT systems constitute two particularly useful systems for site specific integration of transgenes. In these systems, a recombinase (Cre or FLP) will interact specifically with its respective site-specific recombination sequence (lox or FRT, respectively) to invert or excise the intervening sequences. The sequence for each of these two systems is relatively short (34 bp for lox and 47 bp for FRT) and therefore, convenient for use with targeting vectors.

In preferred embodiments, the present disclosure concerns the general methodology for preparing transgenic non-human animal lines. A summary of the general methodology and protocols for the preparation of several different useful transgenic non-human lines is summarized below. The method generally includes first preparing a group of transgenic animals having incorporated into their genome at least one selected transgene, selecting at least one founder from the group of transgenic animals, and breeding the founder or founders to establish transgenic animals having the selected transgene incorporated into their genome. In the present disclosure, the selected transgene(s) encode a non-eukaryotic expression system, preferably a bacteriophage RNA polymerase, which is operably linked to a tissue-specific and inducible promoter.

C. Methods of Making Transgenic Animals

In general, a transgenic animal is produced by the integration of a given transgene into the genome of the animal in a manner that permits the expression of the transgene. Methods for producing transgenic animals are generally described by Wagner and Hoppe (U.S. Pat. No. 4,873,191; which is incorporated herein by reference); and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety). Transgenic animals may be produced from germ cells or fertilized eggs of a number of animals including, but not limited to reptiles, amphibians, birds, mammals, and fish. The process for generating transgenic animals does not present any difficulties to the skilled person who is familiar with the techniques of microinjection, removal and implantation of embryos, as well as animal husbandry.

Typically, the initial group of transgenic animals are prepared by introduction of DNA that includes the selected transgene into germ cells of the animal (typically fertilized eggs). The transfected germ cells are then employed to generate a transgenic animal. Introduction of the DNA into the germ cells is most conveniently achieved by a technique known as microinjection, although a variety of methods for introducing DNA into germ cells are well known to those of skill in the art. Microinjection involves introducing a transgene, for example in solution, into a germ cell (for example the mature sperm, egg, or polar body) using a microscope and a microinjector pipet, which deposits intact DNA into pronuclei. The transgene may be microinjected into the nucleus or pronucleus of the zygote or into the nucleus of a primordial germ cell that is diploid (e.g., a spermatogonium or oogonium). This gamete will preferably form a zygote or the pronucleus of the zygote. Prior to introduction of the transgene, the germ cell may be induced to undergo decondensation to facilitate the incorporation of the exogenous material. Decondensation techniques are described by Mahi et al., (1975) J Reprod. Fert. 44:293-296, Hendricks et al., (1965) Exptl. Cell Res. 40:402-412, and Wagner et al., (1978) Arch. Androl. 1:31-41, each incorporated herein by reference. Examples of other techniques that may be employed to introduce DNA into the genome are in vitro fertilization using sperm as a carrier of exogenous DNA, and electroporation or transfection of the transgene into an embryonic stem cell line, followed by introduction of these cells into a blastocyst.

The transgene can be prepared for microinjection by any means known in the art. For example, a transgene construct may be cleaved with enzymes appropriate for removing the bacterial plasmid sequences, and the DNA fragments electrophoresed on agarose gels in TBE buffer, using standard techniques. The DNA bands are visualized by staining with ethidium bromide, and the band containing the transgene is excised. The excised band is then purified from the gel using methods well known to those of skill in the art, and the transgene is isolated for microinjection. Methods for purification of DNA for microinjection are described in Hogan et al., Manipulating the Mouse Embryo (Cold Spring Harbor Laboratory, Cold Spring Harbor, N.Y., 1986); Palmiter et al., (1982)Nature 300:611; The Qiagenologist, Application Protocols, 3rd edition, published by Qiagen, Inc., Chatsworth, Calif; and Sambrook et al., (1989), all of which are incorporated by reference herein.

In an exemplary microinjection procedure for the generation of transgenic mice, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed and embryos are recovered from excised oviducts and placed in Dulbecco's phosphate buffered saline with 0.5% bovine serum albumin (BSA; Sigma). Surrounding cumulus cells are removed with hyaluronidase (1 mg/mL). Pronuclear embryos are then washed and placed in Earle's balanced salt solution containing 0.5% BSA (EBSS) in a 37.5° C. incubator with a humidified atmosphere at 5% CO2, 95% air until the time of injection.

A purified nucleic acid fragment containing the transgene of interest is isolated and resuspended at 50 μg/μl. The resuspended DNA solution is placed on a microscope slide adjacent to the cells to be injected and covered with paraffin oil. Approximately 10 picoliters (pL) of DNA solution is drawn into a 1 μm diameter injection pipet. The injection pipet is then juxtaposed to the nucleus or pronucleus and the cell to be injected. Injected cells are placed in culture tubes for five days before being transplanted into the uteri of foster mothers who are at an appropriate stage of pseudopregnancy. Embryos can be implanted at the two-cell stage. Preimplantation culture conditions are described in Hoppe and Pitts, (1973) Biol. Reprod. 8:420-426, incorporated herein by reference.

Randomly cycling adult female mice are paired with vasectomized males. FVB, C57BL/6NHsd, Swiss mice, or other comparable strains can be used for this purpose. Recipient females are mated at the same time as donor females. At the time of embryo transfer, the recipient females are anesthetized with an intraperitoneal injection of 0.015 mL of 2.5% avertin per gram of body weight. The oviducts are exposed by a single midline dorsal incision. The incision is made through the body wall directly over the oviduct. The ovarian bursa is then torn with watchmakers forceps. Embryos to be transferred are placed in DPBS (Dulbecco's phosphate buffered saline) in the tip of a transfer pipet (about 10 to 12 embryos). The pipet tip is inserted into the infundibulum and the embryos transferred. After the transfer, two sutures close the incision. Implanted embryos are carried to term. A skilled artisan is aware that transgenic mice and rats are also commercially available, for example from DNX Transgenics (Princeton, N.J.) and Charles River Laboratories (Wilmington, Mass.).

Another exemplary procedure for the generation of transgenic animals is a method for generating transgenic rats. Preferred methods for preparing transgenic rats include subjecting a female to hormonal conditions effective to promote superovulation, followed by fertilizing eggs of the superovulated female and introducing the selected transgene into the fertilized eggs (U.S. Pat. No. 4,873,191, incorporated herein by reference). Preferably, fertilized eggs are isolated from outbred Sprague Dawley rats. In this embodiment, the fertilized eggs having the selected transgene are transferred into a pseudopregnant female rat and brought to term. Promoting superovulation in female rats can be accomplished using techniques known to those skilled in the art. For example, superovulation may be effected by intraperitonial injection of pregnant mare chorionic gonadotropin (PMCG) or pregnant mare serum gonadotropin (PMSG) and human chronic gonadotropin (HCG) into female rats. Alternatively, superovulation may be promoted by employing a technique developed by Armstrong and Opavsky, (1988) Biol. Reproduct. 39:511-18 (incorporated herein by reference), where immature rats are superovulated by a continuous infusion of follicle stimulating hormone.

After superovulation is effected and the females are bred, the resultant eggs are collected (by flushing from the oviduct). While the presently preferred method for fertilizing eggs is by breeding the female with a fertile male, superovulated eggs could also be fertilized by artificial insemination of the female, or by in vitro fertilization after removal of the eggs. After fertilization, the donor females are sacrificed and the oviducts are dissected into a buffer, for example Dulbecco's Phosphate Buffered Saline (DPBS, Gibco-BRL) supplemented with 0.5 g bovine serum albumin (Sigma) and antibiotics. The oviducts are placed in a standard culture media and the ampulla section of the oviduct is incised to extract the cumulus masses into the media for the removal of the cumulus cells from the embryos. Embryos are collected and incubated in a suitable media for subsequent microinjection. The selected transgene is introduced into the fertilized egg by a convenient method, for example, microinjection into male pronuclei of the fertilized rat oocytes. It is also possible that the transgene may be introduced into the germ cell prior to fertilization. Next, the fertilized eggs with the selected transgene are transferred into a pseudopregnant recipient female, for example an adult Long-Evans rat, using any of the techniques that are well known in the art, for example, techniques that are typically employed in connection with transgenic mice. See e.g., Brinster et al., (1985) Proc. Natl. Acad. Sci. USA 82:443842, incorporated herein by reference.

A final exemplary procedure for the generation of transgenic animals is a method for generating transgenic rabbits, as previously described by U.S. Pat. No. 6,512,161, and Duverger et al., (1996) Arterioscler. Thromb. Vasc. Biol. 16:1424-1429, each incorporated herein by reference. Briefly, an adult female rabbit is superovulated, and then mated on the same day that luteinizing hormone is injected into the rabbit. Embryos are collected 17 hours later. The transgene is injected into the embryo and transferred to a pseudopregnant female, for example as disclosed in Fan et al., (1995) Arterioscler. Thromb. Vasc. Biol. 15:1889-1899, incorporated herein by reference, and the embryos are carried to term.

Once the fetuses in the pseudopregnant female have been brought to term, a founder animal with the transgene incorporated into its genome is identified by standard techniques known to those of skill in the art, such as monitoring transient expression of reporter genes, detecting hybridization of transgene DNA to genomic DNA from weanling offspring, for example by Southern blot or polymerase chain reaction (PCR), or detecting hybridization of the transgene DNA to mRNA isolated from various tissues by Northern blot. The word “founder” is intended to refer to a transgenic animal that develops from the microinjected egg. Founders may be tested for expression of a functional gene by any suitable assay of the gene product. Founders that express the transgene are then bred to establish a line or lines of transgenic animals that have the selected transgene incorporated into their genomes. The line or lines of transgenic animals may include hemizygous, heterozygous, and homozygous animals. As used herein, the term “progeny” is intended to refer to all offspring and descendents of transgenic animals that express the transgene.

The present disclosure contemplates that the foregoing techniques and other techniques not specifically disclosed herein but well known to those of skill in the art for generating transgenic animals can be successfully employed to develop animal lines having any of a number of genes, DNA segments, or transgenically derived traits introduced into their genomes. As used herein, the term “line” is intended to refer to animals that are direct descendants of one founder and bear a transgene locus stably integrated into their germline. It is contemplated that the foregoing technique can be successfully employed using either inbred or outbred animals strains. Inbred strains or lines may have particular advantages, including, for example, reproducibility from one animal to the next, the ability to transfer cells or tissue among animals, and the ability to carry out defined genetic studies to identify the role of endogenous genes.

It will be appreciated upon practicing the present disclosure that not all transgenic animals that have a non-eukaryotic expression system will be suitable for genetic immunization. The observed suitability for genetic immunization in the transgenic animals may be dependent on the copy number of the transgene in the animal, which may lead to higher expression of, for example, a bacteriophage RNA polymerase in the host. Therefore, a heterozygous transgenic animal with a higher copy number of the transgene may be better suited for genetic immunization then a homozygous transgenic animal with a reduced copy number. One may desire to identify and select a transgenic animal with a relatively high copy number of the selected transgene or transgenes incorporated into its genome, such as at least about 1 copy, or alternatively at least about 2, 3, 4, 5, 10, 15, 20, 25, 30, 35, 40, 45, or 50 copies, or possibly even more copies. Alternatively, one may desire to select a transgenic animal with up to about 2, 3, 4, 5, 10, 15, 20, 25, 30, 35, 40, 45, or 50 copies of the selected transgene or transgene incorporated into its genome. The transgenic animals of the present disclosure may carry the transgene in all their cells, or may carry the transgene in some, but not all of their cells, i.e., mosaic animals. In addition, the transgene may be integrated as a single transgene or in tandem, e.g., head-to-head, head-to-tail, or tail-to-tail tandems.

The present disclosure further provides cross-breeding the transgenic non-human animals disclosed herein with other genetically modified animals such as other transgenic animals, or animals in which selected genes have been inactivated or otherwise modified, or back-crossing the animals to a different strain of the same animal, which are other routes for introducing different genes, alleles, or other natural products into the transgenic animal model. Cross-breeding and backcrossing can also be used to further elucidate the genes, biological molecules, genotypes, or genetic backgrounds that play a role in the enhanced receptivity of the transgenic animals disclosed herein to genetic immunization. Cross-breeding and back-crossing can also allow those of skill in the art to examine the suitability of different animal strains and cross-strains for enhanced genetic immunization receptivity, for example different strains of the ImmunoMouse. In addition, transgenic animals can be bred with other transgenic animals where the two transgenic animals express different transgenes, to test the effect of one gene product on another gene product or to test the combined effects of two or possibly more gene products.

For example, the ImmunoMouse of the present disclosure can be bred with a mouse that produces fully human antibodies, for example as disclosed in U.S. Pat. Nos. 5,545,806, 5,661,016, 6,150,584, and 6,673,986, each incorporated herein by reference, to produce a mouse with enhanced responsiveness to genetic immunization that also produces fully human antibodies. The mouse produced by this breeding will have a non-eukaryotic expression system, for example by expressing a bacteriophage RNA polymerase in one or more tissues, and have an immune system in which the mouse antibody genes are inactivated and functionally replaced with human antibody genes. The transgenic mouse may also express an isotype of any one of a number of fully human IgG antibodies (e.g., IgG₁, IgG₂, IgG₄).

D. The ImmunoMouse

The ImmunoMouse is a transgenic mouse model with a transgene encoding a bacteriophage RNA polymerase, for example T7 RNA Polymerase, incorporated into its genome. The ImmunoMouse preferably expresses the bacteriophage RNA polymerase under the control of an inducible and tissue-specific (e.g., liver-specific) promoter. Expression of bacteriophage RNA polymerase in the ImmunoMouse allows the mouse to generate an immune response to genetic immunization using a polynucleotide construct that expresses an immunogen under the control of a promoter recognized by the expressed bacteriophage RNA polymerase. Mouse strains that may be utilized in the present disclosure include those strains that are well known to those of skill in the art, including but not limited to 129, 129S7, A, AKR, FVB, C57BL/6BL, C57BL/6NHsd, C57BL/6BL/10, C57BL/6J, C57BL/6BR/CD, BALB/c, C3H, CBA, DBA/1, DBA/2, HRS/J, C57BL/6L/J, RIIIS/J, SJL, SWR, or other comparable strains, as well as hybrids thereof, for example C57BL/6B6/SJL hybrid.

The suitability of different mouse strains and cross-strains for generating transgenic mice expressing bacteriophage RNA polymerase can be routinely evaluated by those skilled in the art using the techniques described herein. It is further contemplated that inbred or isogenic ImmunoMouse lines can be developed from established inbred or isogenic murine strains.

E. Genetic Immunization

After the eukaryote host with a non-eukaryotic expression system is generated, a polynucleotide construct encoding one or more immunogens is administered to the eukaryote host to elicit an immune response. When the non-eukaryotic expression system of the host recognizes the polynucleotide construct, expression of the immunogen occurs. One advantage of this system is that the polynucleotide construct only drives expression of the immunogen as long as both the non-eukaryotic expression system and the polynucleotide construct are present in the host, thus providing for controlled and transient expression of the immunogen. Preferably the polynucleotide construct will produce strong expression of the immunogen for 48-96 hours in the eukaryote host and then gradually shut expression down. This improved system of genetic immunization has an enormous range of applications (Thalhamer et al., (2001) Endocr Regul. 35(3):143-66, incorporated herein by reference). Not only can the system be used to stimulate and isolate CTLs and antibodies for a specific immunogen, but it can also be used as a genetic vaccine, for example to stimulate mucosal immunity to protect against infectious agents, and as immunotherapy for allergic diseases.

Preferably, the polynucleotide construct encodes an immunogen that will stimulate an antibody-based immune response or cell-mediated immune response that results in production of a specific antibody, antibodies, T-cell helper epitopes, T-cell cytotoxic epitopes, or CTLs that recognize the immunogen. CTLs are important to cell-mediated immune defenses against intracellular pathogens such as viruses and tumor-specific antigens produced by malignant cells. Ideally, the polynucleotide will encode at least one epitope of the immunogen. Preferably the epitope will be about 7 to 20 amino acids, more preferably 8-12 amino acids in length. Typically CTL epitopes will be 8-10 amino acids in length. In addition, a eukaryote host may be immunized with a polynucleotide construct encoding a single immunogen, or may be immunized with multiple polynucleotide constructs encoding different immunogens (“shotgun immunization”) or multiple subunits of a single immunogen that may be too large to be used as a single immunogen.

While the number of immunogens that may be used in the system disclosed herein is nearly limitless, some examples of preferred immunogens include human alpha-1 antitripsen, dust mite allergen, hemagglutinin, proteins from the herpesvirus family (e.g., HSV-1, HSV-2, HHV6, HHV7), the hepatitis family of viruses (e.g., hepatitis A, hepatitis B, hepatitis C, hepatitis C virus core gene hn), and antigens derived from varicella zoster virus, Epstein-Barr virus, cytomegalovirus, poliovirus, rubella virus, dengue virus, rabies virus, mumps virus, measles virus, influenza virus types, and human immunodeficiency virus (e.g., HIV-1, HIV-2, gp 41, gp120, gp130, gp160, gag, pol). The immunogen may also be a tumor antigen (e.g., melanoma associated antigen E, tyrosinases, melanoma antigen recognized by T cells, mutant ras, mutant p53, p97 melanoma antigen, carcinoembryonic antigen), or a protein specific to a particular cancer type, such as an activated oncogene, a fetal antigen, or an activation marker. When the immunogen is specific to a tumor or cancer, it is preferable to design the immunogen so that it will improve the induction of cellular immune responses. Most successful anti-tumor responses are based on cytotoxic T cells rather than antibodies. One example of a method for improving induction of the CTL response over the antibody response is to co-deliver the immunogen encoded by the polynucleotide construct as a fusion protein with ubiquitin (Fu et al., (1998) Vaccine 16:1711-17; Rodriguez et al., (1997) J. Virol. 71:8497-8503, each incorporated herein by reference).

The nucleic acid sequence encoding the immunogen in the polynucleotide construct may be a genomic sequence, a cDNA sequence, or fragments thereof, including but not limited to a sequence that contains additions, deletions, substitutions, silent or conservative mutations, as well as homologous or nearly identical sequences. The nucleic acid sequence may also be a chimeric sequence encoding heterologous sequences, or fusion proteins. The immunogen can be a DNA, RNA, structural RNA, proteins, polypeptides, peptides, or fragments thereof. The nucleic acid sequence, also referred to herein as a polynucleotide, encoding the immunogen can be of animal, mammalian, bacterial, yeast, parasite, or viral origin. In particular embodiments, codon usage in the nucleic acid sequence encoding the immunogen can optimize expression and immunogenicity of the immunogen in the eukaryote host. Several reports have demonstrated improved immunogenicity based on such modifications (Andre et al., (1998) J. Virol. 72:1497-1503; Nagata et al., (1999) Res. Commun. 261:445-51; Uchijima et al., (1998) J. Immunol. 161:5594-99; Vinner et al., (1999) Vaccine 17:2166-75, Chambers and Johnston, (2003) Nature Biotechnol. 21:1088-92, each incorporated herein by reference). Codon usage optimization can be particularly useful when optimizing expression of the immunogen in the eukaryote host derives the immunogen of interest.

The polynucleotide may also encode a polypeptide not found in nature, but which comprises at least one particular epitope of an immunogen that is expressed in such a way that the antigenic potential of the epitope is retained. This may be accomplished by fusing the nucleotide sequence for the epitope to filler sequences that, when expressed, cause presentation of the epitope in a manner similar or identical to that found in the native immunogen molecule. Various methods for engineering epitopes are available and well known to those of skill in the art (Lu et al., (1991) Trends Biotechonol. 9:238-42; Ceman and Sant, (1995) Semin. Immunol. 7:373-87; Moudgil et al., (1998) Immunol. Today 19:217-20; and Malmqvist, (1994) J. Mol. Recognit. 7:1-7, each incorporated herein by reference).

The polynucleotide construct encoding the immunogen is generally placed under the control of (i.e., operably linked to) particular expression sequences such as a promoter, and may also include other regulatory element(s), as disclosed herein. The promoter sequence of the polynucleotide construct used herein is recognized by the non-eukaryotic expression system of the host. Preferably, the non-eukaryotic expression system includes a bacteriophage RNA polymerase that recognizes and binds to the promoter of the polynucleotide construct, thereby driving expression of the immunogen encoded therein. For example, if the host expresses T7 RNA Polymerase, the polynucleotide construct has a promoter that is recognized by T7 RNA Polymerase, for example a T7 RNA Polymerase promoter. The bacteriophage RNA polymerase will bind to and synthesize a transcript from the polynucleotide construct, which is then translated by the cellular machinery into the immunogen. Additional sequences, such as leader sequences, eukaryotic ribosomal recognition sequences, exons, introns, UTRs, polyadenylation sequences, termination sequences (e.g., stop codons), and the like can also be added to the polynucleotide construct to increase message stability, translation, or polypeptide/protein stability. The immune response elicited by the immunogen can be modulated via translation, processing, stability, and presentation of the immunogen.

The promoter is linked to the nucleic acid sequence encoding the immunogen using standard molecular biological techniques as disclosed herein. For example, polynucleotide constructs are typically grown in bacteria such as E. coli and then isolated using standard molecular biology methods. Although, customized vectors can be produced de novo for generating the polynucleotide construct, there are many commercially available cloning vectors that contain common bacteriophage RNA Polymerase promoters. A “cloning vector” is a plasmid or phage DNA or other DNA sequence that is able to replicate autonomously in a host cell. Preferably the cloning vector contains at least one or more restriction endonuclease recognition sites into which a heterologous DNA sequence can be introduced without the loss of an essential biological function of the vector. The vector may replicate autonomously or integrate into the genome of the host cell. The vector may also contain a marker suitable for identifying cells transformed with the cloning vector. Suitable markers include but are not limited to antibiotic resistance (e.g., ampicillin or tetracyclin), green fluorescent protein, β-lactamase, and the like. Some examples of commercially available cloning vectors are pBluescript (Stratagene) and pCR 4Blunt-TOPO (Invitrogen) which contain the T3 and T7 RNA Polymerase promoters, and pGEM-11Zf vector (Promega) that contains the T7 and SP6 RNA Polymerase promoters.

After the polynucleotide construct is generated, it may be administered to the eukaryote host either as a linear fragment or a circular plasmid. The polynucleotide construct may be presented to the eukaryote host either as naked DNA, or it may be administered in an excipient or vehicle such as water, saline, glycerol, ethanol, and the like. Typically the polynucleotide construct composition is prepared as an injectable, either as a liquid solution or suspension, or alternatively as a solid form suitable for solution or suspension in a liquid vehicle prior to injection. In addition, auxiliary substances such as wetting or emulsifying agents, or pH buffering substances, may be present. In other embodiments, a carrier may be included with the polynucleotide construct that does not cause an immune response harmful to the organism receiving the construct. Suitable carriers are well known to those of skill in the art, and include but are not limited to proteins, polysaccharides, polylactic acids, polyglycollic acids, polymeric amino acids, amino acid copolymers, lipid aggregates (such as oil droplets or liposomes), and inactive virus particles.

For example, the polynucleotide construct may be encapsulated in lipids prior to delivery. In general, lipid encapsulation is accomplished using liposomes which are able to stably bind or entrap and retain polynucleotide constructs (Hug and Sleight, (1991) Biochim. Biophys. Acta. 1097:1-17; Straubinger et al., in Methods of Enzymology (1983), Vol. 101, pp. 512-527, each incorporated herein by reference). Methods for preparing liposome-nucleic acid complexes are well known to those of skill in the art (Straubinger et al., in Methods of Immunology (1983), Vol. 101, pp. 512-527; Szoka et al., (1978) Proc. Natl. Acad. Sci. USA 75:4194-4198; Papahadjopoulos et al., (1975) Biochim. Biophys. Acta 394:483; Wilson et al., (1979) Cell 17:77; Deamer and Bangham, (1976) Biochim. Biophys. Acta 443:629; Ostro et al., (1977) Biochem. Biophys. Res. Commun. 76:836; Fraley et al., (1979) Proc. Natl. Acad. Sci. USA 76:3348; Enoch and Strittmatter, (1979) Proc. Natl. Acad. Sci. USA 76:145; Fraley et al., (1980) J. Biol. Chem. 255:10431; Szoka and Papahadjopoulos, (1978) Proc. Natl. Acad. Sci. USA 75:145; and Schaefer-Ridder et al., (1982) Science 215:166, each incorporated herein by reference).

The ratio of condensed DNA to lipid preparation can vary but is generally around 1:1 (mg DNA:micromoles lipid), although a higher ratio of lipid may also be appropriate. Appropriate liposomal preparations include but are not limited to cationic (positively charged), anionic (negatively charged), and neutral preparations. Cationic liposomes are preferred because they have been shown to mediate intracellular delivery of plasmid DNA (Felgner et al., (1987) Proc. Natl. Acad. Sci. USA 84:7413-7416, incorporated herein by reference); mRNA (Malone et al., (1989) Proc. Natl. Acad. Sci. USA 86:6077-6081, incorporated herein by reference); and purified transcription factors (Debs et al., (1990) J. Biol. Chem. 265:10189-10192, incorporated herein by reference), in functional form. The liposomes can comprise multilamellar vesicles (MLVs), small unilamellar vesicles (SUVs), or large unilamellar vesicles (LUVs). The liposomes may have target specific ligands or markers on them to direct them to specific tissues or organs, such as brain, skeletal muscle, heart, pancreas, liver, kidney, spleen, etc.

The polynucleotide construct may be introduced into the eukaryote host through a variety of routes, including but not limited to intravenous, oral, intrapulmonary, subcutaneous, intraperitoneal, transdermal, intradermal, and intramuscular. The polynucleotide construct can be directly injected, for example into a peripheral lymph node (Maloy et al., (2001) Proc. Natl. Acad. Sci. USA 98:3299-3303, incorporated herein by reference), of the host, or alternatively introduced into the host using biolistic technology (gene gun) (Tuting, (1999) Curr. Opin. Mol. Ther. 1(2):216-25, Haynes et al., (1996) J. Biotechnol. 44(1-3):37-42, each incorporated herein by reference), for example the helium-powered Helio Gene Gun delivery system (Bio-Rad Laboratories, Richmond, Calif.). The polynucleotide construct may also be packaged in a particular virus or retrovirus that targets particular cell types. In a preferred embodiment, the polynucleotide construct is introduced into the transgenic animal through a tail vein injection, which direct the majority of the injected polynucleotide construct (e.g., over 90%) to the liver, thereby enhancing the animal's immune response. The polynucleotide construct can also be introduced ex vivo into cells that have been removed from the host, with the subsequent reintroduction of those cells back into the host.

Typically, about 1-50 μg of polynucleotide construct is administered to the eukaryote host, more preferably about 2, 3, 4, 5, 10, 15, 20, 25, 30, 35, 40, or 45 μg. Alternatively, the polynucleotide construct may be administered at a dosage of about 1 ng/kg to about 10 mg/kg, more preferably about 10 μg/kg to about 1 mg/kg. The polynucleotide construct may be administered in about 0.1 to about 100 cc of solution, more preferably about 1, 5, 10, 20, 30, 40, 50, 60, 70, 80, or 90 cc of solution. The polynucleotide construct can be administered multiple times to a eukaryote host over a selected time period, for example injecting once every 5 to 9 days, more preferably every 7 days, for a 21-45 day time period. After the polynucleotide construct is introduced into the eukaryote host, it can exist as free, autonomously replicating DNA or can integrate into the genome of a cell, in which case it replicates along with the genome. Preferably the polynucleotide construct of the present disclosure does not incorporate into the genome of the host, but rather exists transiently as an autonomous or episomal construct or fragment in one or more cells of the host. Once administered, expression of the immunogen triggers an immune reaction, causing the production of CTLs and/or antibodies.

In another embodiment, the polynucleotide construct is introduced into a cell at the same time as the transgene encoding the non-eukaryotic expression system. For example, a transgene encoding a bacteriophage RNA polymerase operably linked to a tissue-specific, inducible promoter is transfected into a germ cell along with a polynucleotide construct encoding an immunogen, and the germ cell generates a transgenic animal. Expression of the bacteriophage RNA polymerase is then induced in the developed animal by stimulating the transgene promoter, thereby generating production of the immunogen, which elicits an immune response. The system is turned off by removing the stimuli inducing expression of the bacteriophage RNA polymerase.

Preferably a therapeutically effective amount of the immunogen is expressed from the polynucleotide construct in the eukaryote host. The term “therapeutically effective amount” means a sufficient amount of an immunogen is expressed in the eukaryote host to produce an immune response. Preferably the immunogen stimulates a cellular immune response, a humoral immune response, or both. In other embodiments, the therapeutically effective amount is an amount sufficient for therapeutic or prophylactic treatment of the eukaryote host. The therapeutically effective amount for each eukaryote host will vary depending on many factors, including but not limited to the age and general condition of the host, the capacity of the host's immune system to produce antibodies or CTLs, the degree of protection desired, for example from a genetic vaccine, the severity of the condition treated, the particular immunogen selected, the mode of administration of the polynucleotide construct, the selectable or inducible non-eukaryotic expression system present in the host, the introduction of additional factors into the host to modulate the immune response, etc. One of skill in the art will be able to readily determine a therapeutically effective amount for a given eukaryote host using routine experimentation.

Several strategies are well known to those of skill in the art to potentiate the results of the genetic immunization system disclosed herein, for example using different modes and sites of delivery for the polynucleotide construct encoding the immunogen, co-delivery of genes or adjuvant molecules with regulatory or stimulatory properties, and modification of the immunogen encoded by the polynucleotide construct by inserting or deleting cytosolic or endosomal transport signals (Cohen et al., (1998) FASEB J. 12:1611-26, Boyle et al., (1997) Proc. Natl. Acad. Sci. USA 94:14626-31, each incorporated herein by reference). The mode and site of delivery of the polynucleotide construct encoding the immunogen will depend in part on which tissue(s) or organ(s) express the non-eukaryotic expression system in the eukaryote host. For example, in one preferred embodiment T7 RNA Polymerase is specifically and inducibly expressed in the liver of a transgenic mouse, which stimulates expression of an immunogen upon administration of the polynucleotide construct to the mouse by tail vein or hypo-osmotic injection. Other examples of useful systems for genetic immunization include, but are not limited to, expression of the non-eukaryotic expression system in skeletal muscle with subdermal administration of the polynucleotide construct, as well as expression in kidney, heart, pancreas (which can be induced with insulin), and spleen (which can be induced with cytokines) with intravenous injection of the construct.

Co-delivery of genes or adjuvant molecules with regulatory or stimulatory properties can also be used to stimulate the immune response to a particular antigen. Modulating the immune response stimulated by the immunogen encoded by the polynucleotide construct can be accomplished by including additional information, for example an immunostimulating agent such as an adjuvant, along with the polynucleotide construct. The additional information may be introduced into the eukaryote host in several ways, for example, by encoding the additional information in a separate cassette in the polynucleotide construct, by co-injecting a second plasmid or recombinant molecule that contains the immunostimulating information with the polynucleotide construct, or by introducing the polynucleotide construct to the host along with one or more adjuvant substances. The polynucleotide construct may be co-delivered together or separately but closely spaced in time with additional information. In addition, suitable carriers may function as immunostimulating agents, or to modulate immune responses. The polynucleotide construct preparation may also be emulsified or encapsulated in liposomes for an enhanced adjuvant effect.

Adjuvant substances suitable for administration with the immunogen include but not limited to cytokines, colony stimulating factors, adhesion molecules, and other stimulatory molecules (Thalhamer et al., (2001) Endocr Regul. 35(3):143-66, incorporated herein by reference). Other examples of suitable adjuvants are aluminum salts (e.g., aluminum hydroxide, aluminum phosphate, aluminum sulfate); oil-in-water emulsion formulations (with or without other specific immunostimulating agents); saponin adjuvants (e.g., Stimulon, ISCOMs (immunostimulating complexes)); Complete Freunds Adjuvant (CFA) and Incomplete Freunds Adjuvant (IFA); cytokines (e.g., interleukins (IL-1, IL-2, IL-7, IL-12, IFN-γ), macrophage colony stimulating factor (M-CSF), granulocyte-macrophage colony-stimulating factor (GM-CSF), FMS-like tyrosine kinase 3 ligand (Flt3L), tumor necrosis factor (TNF), etc.); leucocyte surface molecules (e.g., CD28, CD80, CD86, cytotoxic T-lymphocyte-associated protein 4 (CTLA4)); CpG DNA motifs; intracellular adhesion molecule-1 (ICAM-1); lymphocyte function associated-3 (LFA-3), vascular cell adhesion molecule-1 (VCAM-1); third complement protein (C3d); and other substances which are well known to those of skill in the art to act as immunostimulating agents. The polynucleotide construct can also be co-delivered with epitopes that recruit T helper cells, for example by fusing exogenous T-cell epitopes to the immunogen (King et al., (1998) Nat. Med. 4:1281-86; Dalum et al., (1996) J. Immunol. 157:4796-4804, each incorporated herein by reference).

In other embodiments, the immunogen is engineered to express the immunogen in different cellular locations for better immunogen presentation, thereby potentiating the host's immune response to the desired antigen (Aberle et al., (1999) J. Immunol. 163:6756-61; Boyle et al., (1997) Int. Immunol. 9:1897-1906, each incorporated herein by reference). Form of the immunogen may play an important role in the induction and efficiency of the immune response. For example, a given immunogen may be highly immunogenic in one form and poorly or non-immunogenic in another form (Leitner et al., (1997) J. Immunol. 159:6112-19; Weiss et al., (1999) Vaccine 18:815-24). Therefore, the immunogen encoded by the polynucleotide construct may be designed to be a secreted, membrane-bound, or cytoplasmically sequestered immunogen, depending on which forms elicits the desired immune response.

Another way to enhance the immunogenicity of an epitope-coding sequence, such as a single T or B cell epitope of an antigen, is to fuse the sequence with an ER-targeting leader sequence (Ciernik et al., (1996) J. Immunol. 156:2369-75; Hsu et al., (1998) Int. Immunol. 10:144147, each incorporated herein by reference). This antigen may then circumvent the proteasome (and degradation of the antigen) by direct translation of a signal sequence plus peptide into the ER. Protein solubility of the immunogen can also be improved, for example, by including highly soluble and stably folded domains in the immunogen (Chambers and Johnston, (2003) Nat. Biotech. 21:1088-92, incorporated herein by reference). All of the methods disclosed herein for optimizing the immune response of the eukaryote organism to the immunogen encoded by the polynucleotide construct also apply when the polynucleotide construct is used as a vaccine.

The polynucleotide construct encoding the immunogen can also be used as a genetic vaccine, either as a prophylactic vaccine (to prevent infection) or a therapeutic vaccine (to treat disease after infection). The polynucleotide construct may be used individually or in combination, in vaccine compositions. The vaccines as contemplated herein can comprise mixtures of one or more antigens, such as glycoproteins derived from more than one viral isolate. The vaccine may also be administered in conjunction with other immunoregulatory agents, for example, immunoglobulins, cytokines, lymphokines, and chemokines, including but not limited to IL-2, GM-CSF, IL-12, γ-interferon, IP-10, MIPβ, and RANTES.

F. Antibodies and CTLs

The present disclosure is directed to stimulating production of antibodies or CTLs by genetic immunization in a eukaryote host to a selected immunogen. These antibodies or CTLs can be harvested using conventional means well known to those of skill in the art. Methods for isolating polyclonal antibodies or obtaining continuous stable production of monoclonal or single-chain antibodies are well known in the art (Barry et al., (1994) Biotechniques 16:616-19; Chowdhury et al., (1998) Proc. Natl. Acad. Sci. USA 95:669-74, each incorporated herein by reference). For example, polyclonal antisera can be obtained by bleeding the immunized animal into a glass or plastic container and incubating the blood at 25° C. for one hour, followed by incubating the blood at 4° C. for 2-18 hours. Next, the serum is recovered by centrifugation. Generally, this method will isolate polyclonal antibodies. Each transgenic animal yields serum sufficient for hundreds of immunoassays.

Monoclonal antibodies (MAbs) can be prepared, for example, using the method of Kohler and Milstein, (1975) Nature 256:495-97 (incorporated herein by reference), or modifications thereof. Typically, a mouse or rat is first immunized with injections of the immunogen. However, rather than bleeding the animal to extract the serum, the spleen (and optionally several large lymph nodes) is removed and dissociated into single cells. If desired, the spleen cells can be screened (after removal of nonspecifically adherent cells) by applying a cell suspension to a plate or well coated with protein antigen. B-cells producing membrane-bound immunoglobulin specific for the antigen bind to the plate, and are not rinsed away with the rest of the suspension. The resulting B-cells are then induced to fuse with myeloma cells to form hybridomas, and are cultured in a selective medium (e.g. hypoxanthine, aminopterin, thymidine medium; “HAT”). The resulting hybridomas are plated by limiting dilution, and are assayed for the production of antibodies that specifically bind to the immunizing antigen (and which do not bind to unrelated antigens). The selected MAb-secreting hybridomas are then cultured either in vitro (e.g. in tissue culture bottles or hollow fiber reactors) or in vivo (as ascites in mice). Other methods for sustaining antibody-producing B-cell clones, such as by EBV transformation, are known.

If desired, the antibodies (whether polyclonal or monoclonal) may be labeled using conventional techniques. Suitable labels include but are not limited to fluorophores, chromophores, radioactive atoms (particularly 32P, 125I, 131I, 111In and 90Y), electron-dense reagents, enzymes, and ligands having specific binding partners. Enzymes are typically detected by their activity. For example, horseradish peroxidase is usually detected by its ability to convert 3,3′,5,5′-tetramethylbenzidine (TNB) to a blue pigment, quantifiable with a spectrophotometer.

The antibodies or CTLs produced according to the presently disclosed genetic immunization system can be used to vaccinate or treat a patient for disease by administering an effective dose of the antibodies or CTLs produced as described above. Historically, some diseases and ailments, like rabies, diphtheria, snake-bite, botulism and hepatitis A have been prevented or treated with antibody injections. Today, antibody administration as a disease treatment is becoming more commonplace. Recently, administration of antibodies have been used to treat various cancers such as breast cancer (Perez and Hortobagyi, 2000, Semin Oncol 6 Suppl 11:26-32), melanoma (Shaw et al.,1997, Head and Neck 19:595-603), asthma (Easthope and Jarvis,2001, Drugs 61:253-60) and HIV (Yang et al.,1995, J Mol. Biol. 254:392-403), to name but a few. The system disclosed herein provides improved methods to develop additional antibodies, CTLs, and vaccines utilizing genetic immunization.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Production and Analysis of ImmunoMouse Expressing T7 RNA Polymerase

The ImmunoMouse model that expresses T7 RNA Polymerase was generated by constructing a transgene encoding T7 RNA Polymerase operably linked to an ApoAI promoter, and subsequently incorporating the transgene into the genome of a mouse. The sequence encoding T7 RNA Polymerase was derived from pAR1173, which contains the complete coding sequence of the gene for bacteriophage T7 RNA polymerase (T7 gene 1) cloned into the plasmid pBR322 as reported by Davanloo et al., (1984) Proc. Natl. Acad. Sci. USA 81(7):2035-39, incorporated herein by reference. The full length T7 RNA Polymerase transgene was constructed by digesting pAR1173 with BamHI, treating the digested plasmid with Klenow, and purifying the 2.7 kb BamHI fragment encoding T7 RNA Polymerase.

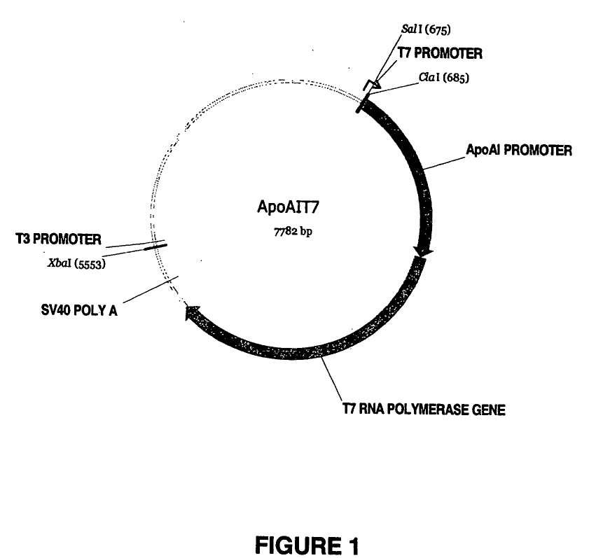

The 2.7 kb BamHI fragment was next sub-cloned into a plasmid that contained the apolipoprotein AI (ApoAI) promoter and an SV40 intron with a polyA termination sequence. The plasmid was derived from the L4-1 plasmid, which was designed to express the cDNA for cholesteryl ester transfer protein (CETP), with the ApoAI promoter upstream of the cDNA and the SV40 intron downstream of the cDNA. To remove the CETP cDNA from the LA-1 plasmid, and substitute the nucleic acid fragment encoding T7 RNA Polymerase, the L4-1 plasmid was digested with HindIII and EcoRI, treated with Klenow and SAP, and gel purified away from the released 1.5 kb CETP cDNA fragment. The 2.7 kb fragment encoding T7 RNA Polymerase was ligated with the digested and purified L4-1 plasmid, and colonies were screened for plasmids with the 2.7 kb fragment encoding T7 RNA Polymerase functionally inserted downstream of the ApoAI promoter and upstream of the SV40 intron, as illustrated in FIG. 1. After transgene plasmids with the T7 RNA Polymerase fragment inserted in proper orientation and frame were isolated, the transgene was prepared for microinjection. The T7 RNA Polymerase transgene fragment for microinjection was released and isolated from the transgene plasmid by digesting the plasmid with SalI and XbaI, and isolating the approximately 4.7 kb fragment for microinjection.

The inventors contracted with Chrysalis DNX Transgenics (Princeton, N.J.) to produce transgenic mice expressing T7 RNA Polymerase from the transgene described above. The mice were prepared according to the standard protocol of DNX Transgenics using DNA pronuclear microinjection as described in Hogan, Constantini and Lacy, (1986) Manipulating the Mouse Embryo, A Laboratory Manual, (Cold Spring Harbor Press) and U.S. Pat. No. 4,873,191, each incorporated herein by reference. The transgene was injected into C57BL/6B1 mouse eggs to produce transgenic mice. Three mice were identified from the initial microinjection as integration positive for the T7 RNA Polymerase transgene by Southern blot analysis. Southern blot analysis of genomic DNA isolated from offspring using the 2.7 kb BamHI T7 RNA Polymerase fragment described above demonstrated that some of the offspring were positive for the transgene, as indicated by the presence of the expected 3.8 kb BamHI fragment of the transgene. These mice were bred, and offspring were examined for presence of the transgene.

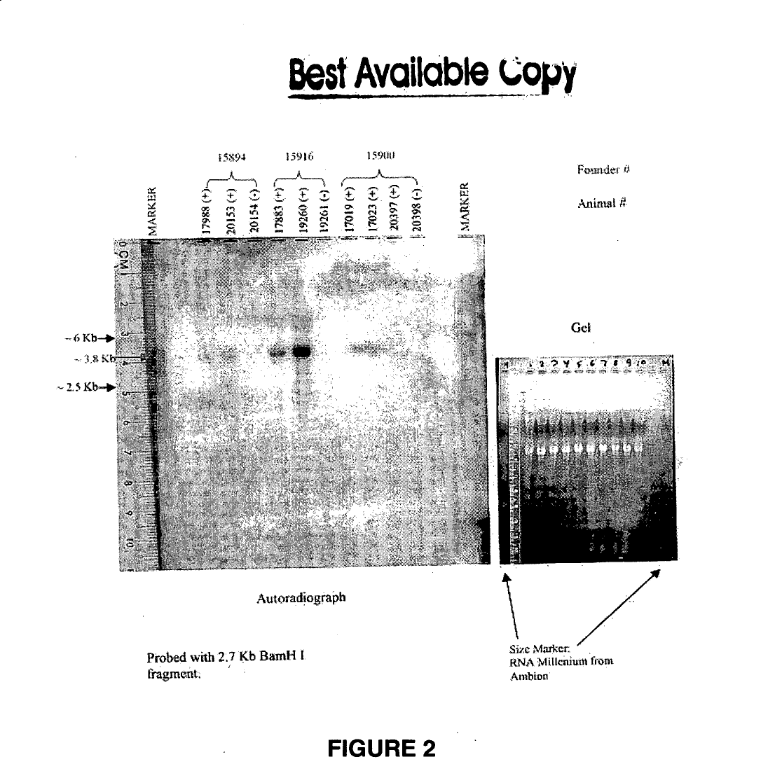

Two founder strains of mice, designated as 15894 and 15916, were established and maintained over several generations by breeding to C57BL/6/BL females (non-transgenic, wild type) with offspring pups examined for the presence of the T7 RNA Polymerase transgene (FIG. 2). As shown in FIG. 2, offspring from one founder (15916) had many copies of the transgene inserted in their genome, while offspring from another founder (15894) had fewer copies of the transgene present in their genome. This result indicates stable integration of the transgene in these mice strains. While the first strain has been become the strain of choice for genetic immunization experiments, the second strain has also been maintained as a backup source of the ImmunoMouse. Tissue-specific expression of T7 RNA Polymerase was also analyzed in the strains. Pups are routinely tested for the genomic presence of the transgene as described above and transgene positive animals are used for antibody development.

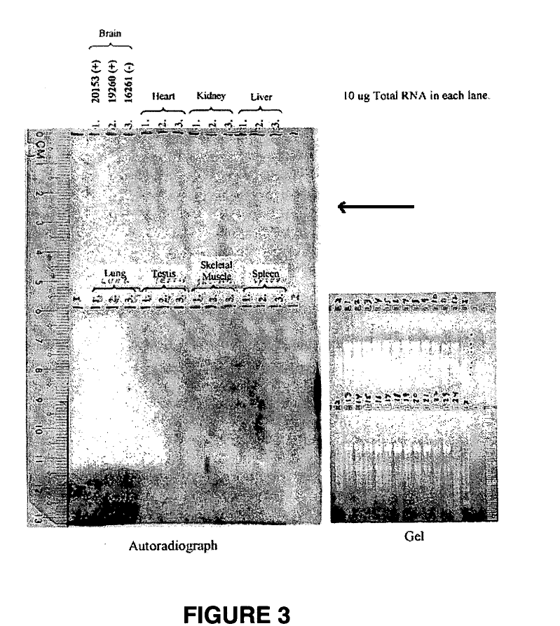

RNA expression of T7 RNA Polymerase in the ImmunoMouse was examined by Northern Blot analysis to determine whether T7 RNA Polymerase was expressed in a tissue specific manner. Total RNA was isolated from heart, lung, liver, kidney, testis, spleen and skeletal muscle of these mice and transferred to nylon membrane using established procedures (Sambrook et al. 1989, Molecular Cloning: A Laboratory Manual 2^{nd} Ed. Cold Spring Harbor Laboratory Press, Cold Spring Harbor, N.Y., specifically incorporated herein by reference) and probed with radiolabeled T7 RNA polymerase riboprobe. The results shown in FIG. 3 demonstrated that the ImmunoMouse strains generated specifically express T7 RNA Polymerase in the liver, and have no detectable expression of T7 RNA Polymerase in other tissues examined, including brain, heart, kidney, lung, testis, skeletal muscle, and spleen.

The examples that follow describe experiments done with the ImmunoMouse animals to demonstrate that these mice have enhanced receptivity to genetic immunization compared to control mice. The inventors later learned, however, that the colonies of ImmunoMouse used in the examples below were infected with Helicobacter hepaticis, a bacterial infection of the liver. The consequence was that while sick ImmunoMouse animals retained the characteristics for enhanced response to DNA immunization, they had unusually high background values to control serum in routine immunoassays and may have had a reduced ability to generate antibodies in response to the selected immunogen. Healthy ImmunoMouse animals have normal background serum values in routine immunoassays and may have an even greater immune response to immunogens. The ImmunoMouse colonies have since been reconstituted, and now have been certified as pathogen-free. The infection was discovered after the experiments set forth in Examples 2 and 3. Experiments 4 through 7 used pathogen-free animals. Initial results indicate that the healthy ImmunoMouse has an even greater ability to generate an immune response through genetic immunization then Examples 2 and 3 would suggest.

### Example 2

Immunization of ImmunoMouse with β-Galactosidase

The first study to determine whether the ImmunoMouse has the ability to produce antibodies in response to genetic immunization was conducted using the bacterial protein β-galactosidase as the test immunogen. To begin, a polynucleotide construct encoding β-galactosidase operably linked to a T7 RNA Polymerase promoter was generated (pCGI-βgal). The vector is composed of a T7 promoter, transcription start sequence, T7 terminator, and β-galactosidase coding sequence in a pET vector. The pCGI vector, which lacked the β-galactosidase gene, was used as a control. Mice received 50 nanograms total DNA per immunization prepared by adding 1 μg of DNA in a volume of 14.4 μl into 34 μl sterile saline followed by adding 2.5 μl Lipofectamine reagent (5:1, DNA:Lipid, Invitrogen) in 47.5 μl sterile saline and mixing well. The mixture was allowed to stand for 45 minutes at ambient temperature prior to use and then diluted to a final volume of 2 mL with sterile saline. Mice received injections via the tail vein of 100 μl of the DNA-Lipid complex. The non-T7 RNA polymerase mice received DNA similarly prepared as above, however the plasmid contained the promoter for CMV to enable eukaryotic expression of the β-galactosidase gene (CMV-βgal). Several mice were also immunized with β-galactosidase protein (5 μg each) as a positive control.

Serum from the immunized mice was initially evaluated in microwell plates (Maxisorp, Nunc) coated with 200 μL per well of β-galactosidase (1 μg/mL) for 2 hours at room temperature and blocked with 0.2% bovine serum albumin (BSA). Serum samples were diluted to 1:100 in Phosphate buffered saline (PBS) and incubated for 45 minutes at room temperature. Bound antibody was detected using a goat anti-mouse IgG HRP conjugate (Kirkegaard & Perry Laboratories, Inc., Gaithersburg, Md.) diluted to 1:500 and incubation with LumiGLO (Kirkegaard & Perry Laboratories). Antibody was also detected by Western immunoblot analysis following SDS-PAGE electrophoresis of 1 μg of β-galactosidase and transfer to nitrocellulose using established procedures. The nitrocellulose was blocked with 1% BSA and serum samples (1:100 dilution) were incubated with the membrane for 2 hours. Following extensive washing the bound antibody was detected with goat anti-mouse IgG and LumiGLO reagents (Kirkegaard & Perry Laboratories).

Testing of the antisera showed that the mice that were immunized with pCGI-βgal and contained the transgene showed a positive response for anti-β-galactosidase antibodies. The response was detected in both an ELISA and Western Blot format. The observed response was superior to mice immunized with the CMV promoter operably linked to β-galactosidase, and was comparable to mice immunized with protein. Genetic immunization was used to elicit an immune response in four groups of animals, and the results were compared. In the first group, ImmunoMouse transgenic animals were injected with 1 to 5 μg of pCGI-βgal. In the second group, ImmunoMouse transgenic animals were injected with 1 to 5 μg of pCGI. In the third group, non-transgenic Balb/C mice were injected with 1 to 5 μg of CMV-βgal. And in the fourth group, non-transgenic Balb/C mice were injected with β-galactosidase protein (5 μg). Each construct administered to the different groups of mice was injected in 0.1 mL saline containing the cationic lipid Lipofectamine (Invitrogen). The mice were immunized at 7 day intervals for 21 days, and production of antibodies was subsequently analyzed by ELISA. The bleed protocol was 100 μl orbital bleeds on indicated days.

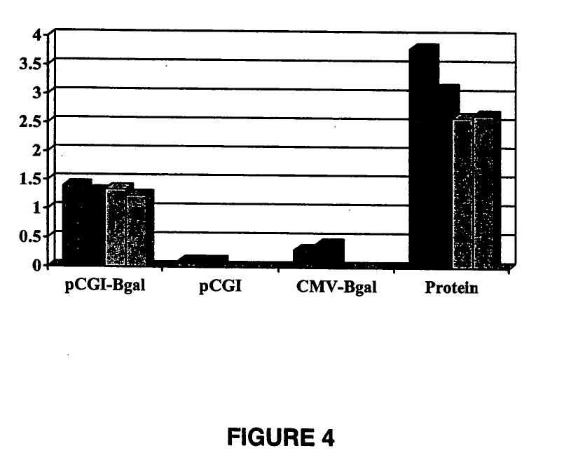

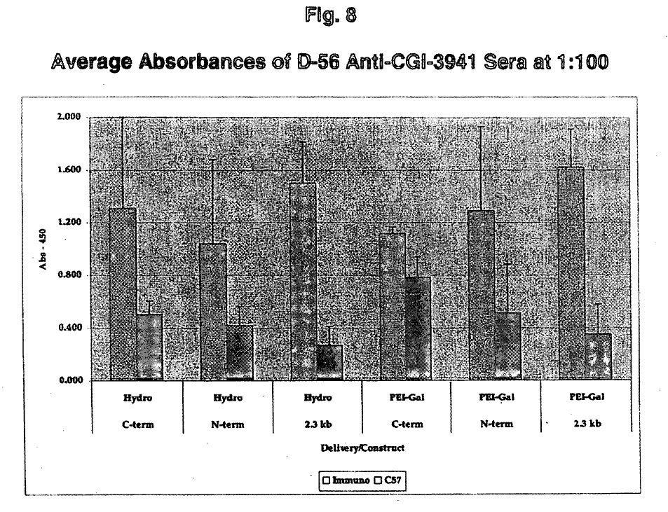

The results, as shown in FIG. 4, demonstrate that ImmunoMouse animals injected with the pCGI-βgal had a strong antibody response that was absent in ImmunoMouse animals injected with pCGI. Balb/C mice injected with CMV-βgal had a poor antibody response, demonstrating the limitations of a traditional genetic immunization construct. Balb/C mice injected directly with β-galactosidase protein had the best immune response of the four groups. Nevertheless, immunization with pCGI-βgal in the ImmunoMouse generated an immune response that was approximately 70% of that observed with direct β-galactosidase protein immunization in Balb/C mice. Another encouraging aspect of this initial experiment was the titration curve, which demonstrated that the decay of antibodies derived from the ImmunoMouse was very similar to the decay observed with protein immunization (FIG. 5). This result is a significant improvement over results with traditional genetic immunization.

### Example 3

Immunization of ImmunoMouse with TIMP-2*

Tissue inhibitor of metalloproteinase 2 (TIMP-2) is a protein associated with breast cancer that has proven to be difficult to produce antibodies against. Thus, TIMP-2 was the next immunogen selected for genetic immunization of the ImmunoMouse. Prior to initiating experiments with TIMP-2, the inventors noticed that older ImmunoMouse animals had greater immune responses after genetic immunization than younger ImmunoMouse animals. This observation suggested that ImmunoMouse animals accumulate sufficient levels of T7 RNA Polymerase for genetic immunization as disclosed herein over time, and the presence of greater levels of T7 RNA Polymerase result in stronger immune responses to immunogens. Since the T7 RNA Polymerase encoded by the transgene was under control of the ApoA1 promoter, the inventors proposed that adding alcohol to the drinking water of the ImmunoMouse colony would result in elevated levels of T7 RNA Polymerase in the mice. Although ApoA1 has not been previously identified as being specifically inducible by alcohol, the inventors speculate that since ApoA1 is a liver-specific promoter the presence of alcohol may induce some increase in expression of the ApoA1 promoter due to an overall increase in liver metabolism. Thus, a group of ImmunoMouse animals received 0.1% alcohol in their drinking water one week prior to genetic immunization.

A polynucleotide construct encoding TIMP-2 operably linked to a T7 RNA Polymerase promoter (pCGI-TIMP) was generated by first amplifying the coding sequence of the TIMP-2 gene. The primer ends were designed to contain BamHI and HindIII ends. The amplified TIMP-2 fragment was digested with BamHI and HindIII and ligated in frame into pET20B+, which contains a T7 polymerase promoter sequence upstream of the BamHI and HindIII sites. The plasmid was transformed into a receptive E. coli strain. Production of TIMP-2 protein was confirmed from E. coli extracts using Western Blotting and a polyclonal TIMP-2 antibody. A control construct that did not encode TIMP-2 but still contained the T7 RNA Polymerase promoter was also generated (pCGI) as set forth in Example 2. Finally, TIMP-2 protein was prepared for administration to control mice. Genetic immunization was used to elicit an immune response in four groups of animals, and the results were compared. In the first group, ImmunoMouse animals were injected with 1 to 5 μg of pCGI-TIMP. In the second group, ImmunoMouse animals were injected with 1 to 5 μg of pCGI. In the third group, Balb/C mice were injected with TIMP-2 protein (5 μg, subcutaneously). And in the fourth group, ImmunoMouse animals that had received 0.1% alcohol in their drinking water one week prior to immunization were injected with pCGI-TIMP. Each construct administered to the different groups of mice was injected in 0.1 mL saline containing the cationic lipid Lipofectamine (Invitrogen). The mice were immunized at 7 day intervals for 21 days, and production of antibodies was subsequently analyzed by ELISA.

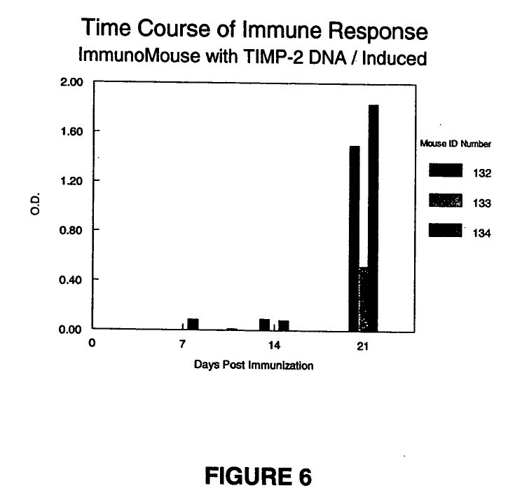

The results demonstrated that while Balb/C mice injected directly with TIMP-2 protein had an immune response, ImmunoMouse animals induced with alcohol and injected with the pCGI-TIMP had the strongest antibody response of any of the groups studied. The control mice had no significant immune response. Thus, the induced ImmunoMouse group out-performed the non-induced ImmunoMouse group, the negative control ImmunoMouse group, and even the putative positive control Balb/C group immunized with TIMP-2 protein. While the difference in response between the induced and non-induced ImmunoMouse groups may not have been statistically significant (due to the limited number of animals tested) the results suggest that alcohol induction does produce some enhancement of expression of the ApoA1 promoter. In addition, ImmunoMouse animals that were induced with alcohol showed a positive immune response to TIMP-2 after 21 days (FIG. 6), as well as a strong response in titer following a boost on day 45.

### Example 4

Comparison of the Immune Response of C57BL/6NHsd and ImmunoMouse Animals Following Injection of DNA Coding for Portions of the CGI-3941 Protein.

Three DNA constructs were prepared. Each of the vector constructs had a T7 promoter, transcription start sequence, and a CGI-3941-protein-fragrnent coding sequence in the pCITE vector (Novagen). CGI-3941 is a proprietary protein that has been implicated in disease, and has been difficult to raise conventional antibodies against. The three vectors vary according to the type of CGI-3941 protein fragment coding sequence it contains: one includes the N-terminal 0.5 kbp of CGI-3941 (N-term), another contains the C-terminal 0.3 kbp (C-Term), and the third the C-terminal 2.3 kbp of the coding region for the CGI-3941 protein (2.3kb). Corresponding antigens were manufactured to each of these gene constructs by Genway, Inc.

Each vector was injected into C57BL/6NHsd or ImmunoMouse animals as described in Table 1. To induce expression of anti-CGI-3941 polyclonal antibodies in mice (Table 1), the DNA was injected into the mice by hydrodynamic injection in saline (Zhang, et al., Gene Ther. 2004, 11(8):675-82) or by low volume injection in polyethylenimine-galactose (PEI-gal) (Q-Biogene). The total DNA delivered to each mouse was 20 μg per injection. For hydrodynamic injection, DNA was mixed with sterile saline at a final concentration of 10 μg/mL. Two mL of the 10 μg/mL solution was injected via the tail vein in approximately 6 seconds. For PEI-gal, the DNA-PEI-gal complex was prepared according the supplier's instructions. Briefly, for injection of five mice, 10 μl of PEI-gal was mixed with 1 mL of a sterile 5% glucose solution. One hundred μg of DNA was added to 900 μl of a sterile 5% glucose solution. Each solution was gently vortexed. The entire volume of the PEI-gal solution in glucose was then added to the DNA solution in glucose and gently vortexed. The DNA-PEI-gal complex was allowed to sit at room temperature for 20-30 minutes before injection.

TABLE 1
Injection of ImmunoMouse animals and C57BL/6NHsd
Mice with CGI-3941 Constructs

| | | Mice | DNA | | | |
|---|---|---|---|---|---|---|
| | | Per | ug/ | | Injection | |
| Group | Strain | Group | mouse | Construct | Volume | Carrier |
| A | Immuno | 2 | C-Term | C-Term | 2.0 | Saline |
| B | Immuno | 4 | N-term | N-term | 2.0 | Saline |
| C | Immuno | 4 | 2.3 kb | 2.3 kb | 2.0 | Saline |
| D | Immuno | 2 | C-Term | C-Term | 0.4 | PEI |
| E | Immuno | 4 | N-term | N-term | 0.4 | PEI |
| F | Immuno | 4 | 2.3 kb | 2.3 kb | 0.4 | PEI |
| H | C57BL/6 | 2 | C-Term | C-Term | 2.0 | Saline |
| I | C57BL/6 | 3 | N-term | N-term | 2.0 | Saline |
| J | C57BL/6 | 4 | 2.3 kb | 2.3 kb | 2.0 | Saline |
| K | C57BL/6 | 2 | C-Term | C-Term | 0.4 | PEI |
| L | C57BL/6 | 4 | N-term | N-term | 0.4 | PEI |
| M | C57BL/6 | 4 | 2.3 kb | 2.3 kb | 0.4 | PEI |

Mice received CGI-3941 immunizations on experimental days 0, 13, 28 and 49. On day 56 the mice were bled and the antibodies present in the sera were compared in an ELISA (Table 2). High-binding immunoassay plates (Dynex) were coated overnight with 4 ug/mL of CGI-3941 protein in phosphate buffered saline (PBS), 100 μl/well. Before use in the assay, the CGI-3941-coated plates were washed three times in ELISA Wash Buffer (Kirkegaard and Perry Labs) and blocked by the addition of 180 μl/well of PBS with 1% chicken serum. Following a 30 minute incubation at room temperature the plates were washed three times. One hundred μL of each sera, diluted 1:100 in 0.5% Tween-20 (PBS-T; Sigma Chemical Company) containing 0.5% chicken serum (PBS-T-CS) was added to duplicate wells. The plates were sealed and incubated at room temperature in a draft-free environment for 60 minutes.

The plates were then washed four times and 95 μL of detection antibody (gamma-specific, peroxidase-labeled goat anti-mouse, diluted 1:1000 in PBS-T-CS) was added to each well. The plates were sealed and incubated for 30 minutes at room temperature in a draft-free environment. The plates were then washed six times and 100 μl of TMB substrate (BioFx) was added to each well. The plates were incubated in the dark at room temperature for 15 minutes and the reactions stopped by the addition of TMB Stop Reagent (BioFx) and the absorbances determined at 450 nm.

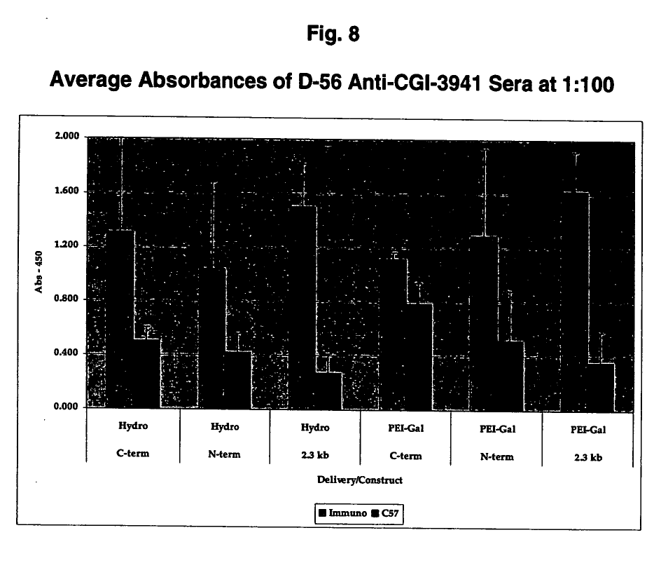

As is shown in FIG. 8, the ELISA showed that the average absorbance for the ImmunoMouse animals in each group was higher than that for the C57BL/6NHsd mice. Based on the results of the ELISA, each of the sera with absorbance values above 1.500 was tested by ELISA titration to determine the individual titers. The ELISA for determining the titer point of individual serum samples was performed as described above, with slight modification for dilution of the sera. The sera were titrated in 96-well round bottom plates: 120 μL of PBS-T-CS was added to the wells in rows B-H of the dilution plate. 200 μL of the appropriate sera, diluted 1:100 in PBS-T-CS, were added to duplicate wells in row A. To prepare serial 1:3 dilutions, 60 μL of sera from the wells in row A were transferred, using a multichannel pipettor, to the corresponding wells in row B. The solutions were mixed by pipetting up and down five times and gently expelling all of the liquid into the wells in row B. The process was repeated down the plate through row G. No sera were transferred to row H. Wells in row H served as assay blanks, containing only buffer. The number of dilution plates used was dependent on the number of sera to be evaluated.

The CGI-3941-coated plates, blocked with PBS with 1% chicken serum, were washed three times. One hundred μL was then transferred from row H of the dilution plate to the corresponding wells of the CGI-3941-coated plate. One hundred μL was then transferred from the dilution plate row G to the corresponding wells of the CGI-3941-coated plate. The procedure was repeated in order for rows F, E, D, C, B and A. The CGI-3941-coated plates with the sera titrations were then sealed and incubated for 60 minutes at room temperature in a draft-free environment.

The plates were then washed four times and peroxidase-labelled goat anti-mouse IgG (gamma), diluted 1:1000 in PBS-T-CS, was added to all wells (95 μL/well). The plates were sealed, incubated for 30 minutes at room temperature and washed six times. One hundred μL of TMB substrate (BioFX) was added to all wells and the plates were incubated in the dark for 15 minutes at room temperature. The reactions were stopped by the addition of TMB Stop Reagent (BioFX). The stop reagent was added in the same order as the substrate. The absorbances at 450 nm were determined using microplate reader and the titers determined as described below.

Seven of the 20 ImmunoMouse animals had absorbances above 1.500. Because none of the C57BL/6NHsd mice met the criteria, the two sera with the highest absorbance values were chosen for inclusion in the titration assay. The results of the titration assay are presented in Table 2.

TABLE 2
Determination of Anti-CGI-3941 Titers for Selected Sera

| | Immuno | Immuno | Immuno | Immuno | Immuno | Immuno | Immuno | C57BL/6NHsd | C57BL/6NHsd |
|---|---|---|---|---|---|---|---|---|---|
| Serum | 3621 | 3617 | 3622 | 3623 | 3625 | 3612 | 3613 | 2932 | 2917 |
| Dilution | Grp A | Grp B | Grp C | Grp C | Grp E | Grp F | Grp F | Grp K | Grp L |
| 100 | 4.000 | 3.945 | 3.909 | 3.986 | 4.000 | 4.000 | 3.946 | 3.107 | 3.435 |
| 400 | 3.266 | 2.908 | 3.252 | 3.601 | 3.911 | 3.628 | 3.510 | 2.130 | 2.567 |
| 1600 | 2.317 | 1.564 | 2.388 | 2.416 | 3.306 | 2.690 | 2.719 | 1.288 | 1.725 |
| 6400 | 1.491 | 0.706 | 1.435 | 1.275 | 2.187 | 1.792 | 1.749 | 0.792 | 1.065 |
| 25600 | 0.800 | 0.323 | 0.649 | 0.578 | 1.101 | 0.874 | 0.913 | 0.420 | 0.555 |
| 102400 | 0.362 | 0.179 | 0.286 | 0.251 | 0.530 | 0.392 | 0.449 | 0.245 | 0.278 |
| 409600 | 0.206 | 0.124 | 0.172 | 0.146 | 0.248 | 0.273 | 0.252 | 0.155 | 0.168 |
| Blank | 0.146 | 0.114 | 0.113 | 0.108 | 0.111 | 0.164 | 0.134 | 0.119 | 0.117 |
| TITER | 17139 | 3980 | 13784 | 11059 | 32714 | 21164 | 22161 | 3579 | 7637 |

The serum titer found at the bottom of Table 2 is defined as the serum dilution required to obtain an absorbance value of 1.000 in the ELISA. The titer is estimated using the formula:
exp(((ln(d)−ln(c))/(b−a)*(tp−a)+ln(c)

where

a=nearest absorbance value above the titer point

b=nearest absorbance value below the titer point

c=serum dilution for a

d=serum dilution for b

tp=titer point.

The titration assay results in Table 2 show that six of the seven ImmunoMouse animals had titers of over 10,000, including three with titers over 20,000 and one with a titer of over 30,000. In contrast, both of the C57BL/6NHsd mice had titers below 7,700. (ELISA results from the 1:100 serum dilution differ from those in FIG. 8 due to the use of a different detection antibody). The results demonstrate that the ImmunoMouse animals consistently showed a significantly better immunization response than the control mice.

The differences between the titer responses of the ImmunoMouse animals and the C57BL/6 mice described in Table 1 were analyzed using the Student's t-test to determine whether or not the two different methods of vector delivery produced statistically significant results. For the 2.3 kbp construct, significant differences were obtained between ImmunoMouse animals and control mice when DNA constructs were injected by either the hydrodynamic injection (P<0.001) or low volume injection (P=0.001) methods. ImmunoMouse animals produced significantly higher titers than control mice with both methods.

Similarly, the difference in titer between ImmunoMouse animals and C57BL/6NHsd mice injected with the N-term construct coupled with PEI-Gal showed a significant difference (P=0.098). A comparison, however, of the ImmunoMouse animals and C57BL/6NHsd control mice exposed to the N-term construct by hydrodynamic injection, gave a P value of 0.126, which indicated that the differences in titer overall were not statistically significant. The inventors speculate that this result may have been an artifact produced by the presence of a single low responder in the ImmunoMouse group.

The sample size for the C-Term vector injections was too small for statistical analysis.

Overall, the results show that ImmunoMouse animals tend to exhibit significantly higher responsiveness than control mice when genetically immunized with CGI-3941 DNA constructs.

### Example 5

Production of Monoclonal Anti-CGI-3941 Antibody.

Mouse 3625 from Example 4 was selected for the production of a monoclonal anti-CGI-3941 antibody. On days 52 and 70 mouse 3625 received an intravenous boost of 20 μg of N-term DNA. On day 73, hybridomas were formed by standard means using PEG 1400 as the fusogen and SP2/0 myeloma cells as the fusion partner. A total of eight×96 well dishes (760 wells with 8 control wells) were planted with hybridomas (equivalent of 2.5×10⁵ splenocytes per well). Ten to eleven days following the fusion, approximately 740 wells contained growing cultures of hybridomas. When tested by ELISA, 71 cultures were selected for expansion and retesting.

Following additional testing, seven cultures were identified which continued to produce anti-CGI-3941. The isotypes for these cultures was determined by ELISA. The results, presented in Table 3, show that five of the cultures produce antibodies of the IgG_{2b} isotype. This is entirely expected, since the serum from mouse 3625, tested in the same assay, also has antibodies predominantly of the IgG_{2b} isotype. However, two of the hybridomas, CH11 and CG1 produced antibodies of the IgG₃ isotype. This was not expected since the serum level of IgG₃ antibodies is quite low and such antibodies are generally associated with carbohydrate or polysaccharide antigens. Subsequently, clones of cultures CH11, CB6 and AA9 were prepared and cryopreserved.

TABLE 3
Isotype Determination of Anti-CGI-3941 Hybridomas
Produced from Mouse 3625

| Culture | | | | | |
|---|---|---|---|---|---|
| ID | Dilution | G1 | G2a | G2b | G3 |
| AA9 | 2 | 0.060 | 0.065 | 0.685 | 0.054 |
| CH11 | 2 | 0.069 | 0.060 | 0.080 | 0.827 |
| CG1 | 2 | 0.065 | 0.070 | 0.088 | 0.362 |
| CB6 | 2 | 0.056 | 0.073 | 1.489 | 0.057 |
| DE4 | 2 | 0.055 | 0.062 | 0.330 | 0.067 |
| GB11 | 2 | 0.055 | 0.065 | 0.578 | 0.062 |
| HG3 | 2 | 0.099 | 0.071 | 0.859 | 0.059 |
| SP2/0 | 2 | 0.060 | 0.069 | 0.089 | 0.063 |
| Buffer | | 0.053 | 0.054 | 0.057 | 0.055 |
| 3625 | | | | | |
| Serum | 4000 | 0.229 | 0.095 | 1.349 | 0.164 |

### Example 6

Comparison of Immune Response of C57BL/6 and ImmunoMouse Animals Following Injection of DNA Coding for CD19.

ImmunoMouse animals and C57BL/6NHsd mice were immunized with DNA coding for CD19, which encodes a cell-surface expressed protein. The construct was placed under the control of a T7 promoter. The types of injections are summarized in Table 4. Mice were injected by either intravenous (IV) or intramuscular (IM) routes. Mice receiving IV injections received either hydrodynamic (2.0 mL) injections or low volume injections (0.2 mL). IM injections were one 0.05 mL injection in each thigh. All injections were in sterile saline. Mice were injected on experimental days 0, 14, 28 and 113.

TABLE 4
Injections of Mice with DNA Coding for CD19

| | | Mice | Injec- | DNA | | | |
|---|---|---|---|---|---|---|---|
| | | Per | tion | ug/ | Injection | | |
| Group | Strain | Group | Route | mouse | Volume | | Carrier |
| A | Immuno | 5 | IV | 40 | 2.0 | mL | Saline |
| B | Immuno | 5 | IV | 20 | 2.0 | mL | Saline |
| C | Immuno | 5 | IV | 10 | 2.0 | mL | Saline |
| D | Immuno | 5 | IM | 2 × 50 | 2 × 0.05 | mL | Saline |
| E | Immuno | 5 | IV | 10 | 0.2 | mL | Saline |
| F | C57BL/6 | 3 | IV | 40 | 2.0 | mL | Saline |
| G | C57BL/6 | 3 | IV | 20 | 2.0 | mL | Saline |
| H | C57BL/6 | 3 | IV | 10 | 2.0 | mL | Saline |
| I | C57BL/6 | 3 | IM | 2 × 50 | 2 × 0.05 | mL | Saline |
| J | C57BL/6 | 3 | IV | 10 | 0.2 | mL | Saline |

Sera from the mice identified in Table 4 were analyzed by FACS using the CD19+ Toledo cell line (ATCC CRL-263 [human b-lymphocytes]) as the target. Sera from four bleed dates were tested at a final dilution of 1:200: prebleed, Jun. 15, 2004 (D-35), Sep. 1, 2004 (D-113) and Sep. 17, 2004 (D-128; 14 days post boost). The data for both mean fluorescence and percentage of gated cells is shown in Table 5. CD19+ Toledo cells (ATCC Catalogue CRL-263) were grown to 0.5-1.0×10⁶ cells/mL in DMEM (high glucose) with 10% fetal bovine serum and 100 units of penicillin and 100 μg of streptomycin per mL. For the assay, the cells were pelletted by centrifugation (1500×g) for 10 minutes at 4° C. and the medium removed. The cells were washed twice by centrifugation in phosphate buffered saline (PBS) without calcium and magnesium. The cells were then resuspended at a concentration of 1×10⁶ cells/mL in PBS and dispensed into 12×75 mm tubes. The cells were again pelleted by centrifugation (900×g) and the supernatant discarded. The cells in each tube were resuspended in PBS and primary antibody (test serum or positive control monoclonal antibody) were added to the appropriate tubes. Monoclonal mouse anti-human CD19 (Biocarta) was used as the positive control antibody.

The antibody-cell suspension was incubated for 30-45 minutes on ice with gentle mixing after 15 minutes. At the end of the incubation period, two mL of PBS was added to each tube to dilute unbound antibodies. The cells were pelleted by centrifugation and washed again with 2 mL of PBS by centrifugation. The cells were resuspended in 1 mL and 5 μL of detection antibody (phycoerytherin-labeled goat anti-mouse IgG Fc; Chemicon) was added to each tube. The reaction mixtures were again placed on ice for 30 minutes, washed three times by centrifugation as above and the cells resuspended in a final volume of 200 μL for FACS analysis.

TABLE 5
FACS Analysis of Anti-CD19 Sera From ImmunoMouse animals and C57BL/6NHsd Mice

| | | | | Mean Values | | | | Pecentage Gated | | | |
|---|---|---|---|---|---|---|---|---|---|---|---|
| | | | | | D-35 | D-112 | D-128 | | D-35 | D-112 | D-128 |
| | | DNA | Inj | | Jun. 15, | Sep. 01, | Sep. 17, | | Jun. 15, | Sep. 01, | Sep. 17, |
| ID | Strain | Dose | Vol | Prebleed | 2004 | 2004 | 2004 | Prebleed | 2004 | 2004 | 2004 |
| 2063 | Immuno | 40 μg | 2 mL | 3.92 | 4.69 | 4.18 | 5.55 | 2.45 | 2.50 | 0.45 | 3.94 |
| 2064 | Immuno | 40 μg | 2 mL | | 5.68 | 5.86 | 7.38 | | 4.97 | 4.25 | 12.68 |
| 2065 | Immuno | 40 μg | 2 mL | 2.97 | 2.79 | 3.48 | 3.33 | 0.16 | 0.16 | 0.11 | 0.13 |
| 2067 | Immuno | 40 μg | 2 mL | 2.76 | 8.68 | 15.74 | 9.91 | 0.02 | 16.92 | 6472 | 28.27 |
| 2066/2258 | Immuno | 40 μg | 2 mL | 3.55 | 21.87 | 5.11 | 5.11 | 0.03 | 61.55 | 3.75 | 2.78 |
| 2243 | Immuno | 20 μg | 2 mL | 2.64 | 4.82 | 6.60 | 7.00 | 0.02 | 1.94 | 9.12 | 10.90 |
| 2244 | Immuno | 20 μg | 2 mL | 3.99 | 4.10 | 5.13 | 5.20 | 1.43 | 1.18 | 2.13 | 2.22 |
| 2245 | Immuno | 20 μg | 2 mL | 11.09 | 5.06 | 5.56 | 5.44 | 30.60 | 2.27 | 0.38 | 0.56 |
| 2246 | Immuno | 20 μg | 2 mL | 5.70 | 5.26 | 8.49 | 8.21 | 4.63 | 1.91 | 19.83 | 17.65 |
| 2248/2343 | Immuno | 20 μg | 2 mL | 2.92 | 4.62 | 3.42 | 4.35 | 0.05 | 0.24 | 0.34 | 1.65 |
| 2086 | Immuno | 10 μg | 2 mL | 7.58 | 6.85 | 4.78 | 5.64 | 13.42 | 8.46 | 2.07 | 3.82 |
| 2089 | Immuno | 10 μg | 2 mL | 2.79 | 3.79 | 3.72 | 5.79 | 0.04 | 1.76 | 1.21 | 3.42 |
| 2090 | Immuno | 10 μg | 2 mL | 6.90 | 4.86 | 3.50 | 3.76 | 10.44 | 3.84 | 0.28 | 0.74 |
| 2091 | Immuno | 10 μg | 2 mL | 3.36 | 5.63 | 3.47 | 7.46 | 0.06 | 3.12 | 0.36 | 13.02 |
| 2092/2344 | Immuno | 10 μg | 2 mL | 3.16 | 4.01 | 5.03 | 18.64 | 0.02 | 0.27 | 4.89 | 70.81 |
| 2249 | Immuno | 100 μg | 0.1 mL | 2.92 | 3.71 | 4.04 | 4.28 | 0.19 | 0.29 | 0.57 | 0.97 |
| 2250 | Immuno | 100 μg | 0.1 mL | 2.79 | 2.99 | 4.66 | 5.51 | 0.07 | 0.19 | 1.45 | 3.08 |
| 2255 | Immuno | 100 μg | 0.1 mL | 2.97 | 4.24 | 4.61 | 36.32 | 0.18 | 0.30 | 0.74 | 88.35 |
| 2257 | Immuno | 100 μg | 0.1 mL | 3.02 | 3.53 | 2.80 | 2.96 | 0.11 | 0.22 | 0.16 | 0.24 |
| 2265 | Immuno | 100 μg | 0.1 mL | 3.22 | 4.94 | 3.78 | 3.85 | 0.06 | 0.53 | 0.40 | 0.60 |
| 2054 | Immuno | 10 μg | 0.2 mL | 2.78 | 5.53 | 4.99 | 8.24 | 0.04 | 4.51 | 2.52 | 17.36 |
| 2055 | Immuno | 10 μg | 0.2 mL | 2.76 | 7.88 | 4.36 | 4.45 | 0.17 | 18.45 | 1.51 | 2.00 |
| 2060 | Immuno | 10 μg | 0.2 mL | 2.77 | 3.03 | 41.92 | 46.14 | 0.01 | 0.04 | 71.35 | 76.05 |
| 2061 | Immuno | 10 μg | 0.2 mL | 3.05 | 5.89 | 5.03 | 5.57 | 0.06 | 2.01 | 2.15 | 4.13 |
| 2057/2365 | Immuno | 10 μg | 0.2 mL | 3.95 | 5.97 | 5.63 | 6.66 | 0.26 | 1.82 | 6.47 | 11.04 |
| 2326 | C57BL/6 | 40 μg | 2 mL | 45.88 | 9.27 | 26.10 | 10.27 | 94.57 | 24.35 | 55.00 | 32.14 |
| 2327 | C57BL/6 | 40 μg | 2 mL | 2.74 | 3.38 | 3.41 | 3.38 | 0.06 | 0.57 | 0.60 | 0.54 |
| 2328 | C57BL/6 | 40 μg | 2 mL | 2.65 | 8.86 | 6.02 | 9.10 | 0.05 | 22.88 | 2.49 | 17.72 |
| 2329 | C57BL/6 | 20 μg | 2 mL | 2.74 | 3.63 | 6.21 | 7.26 | 0.02 | 0.52 | 9.40 | 14.38 |
| 2330 | C57BL/6 | 20 μg | 2 mL | 2.74 | 3.25 | 3.38 | 3.38 | 0.07 | 0.17 | 0.35 | 0.33 |
| 2331 | C57BL/6 | 20 μg | 2 mL | 2.61 | 3.38 | 6.48 | 4.32 | 0.04 | 0.18 | 12.10 | 0.86 |
| 2287/2271 | C57BL/6 | 10 μg | 2 mL | 3.27 | 3.50 | 4.09 | 4.37 | 0.08 | 0.48 | 0.38 | 0.56 |
| 2292/2272 | C57BL/6 | 10 μg | 2 mL | 2.60 | 2.93 | 4.24 | 3.29 | 0.01 | 0.08 | 2.36 | 0.19 |
| 2273 | C57BL/6 | 10 μg | 2 mL | 2.67 | 3.16 | | 2.74 | 0.08 | 0.23 | | 0.13 |
| 2332 | C57BL/6 | 100 μg | 0.1 mL | 3.19 | 3.00 | | 2.92 | 0.14 | 0.19 | | 0.12 |
| 2333 | C57BL/6 | 100 μg | 0.1 mL | 12.12 | 5.13 | | 4.78 | 45.69 | 3.20 | | 1.65 |
| 2334 | C57BL/6 | 100 μg | 0.1 mL | 2.91 | 3.18 | | 4.29 | 0.48 | 0.47 | | 1.60 |
| 2286 | C57BL/6 | 10 μg | 0.2 mL | 2.91 | 3.17 | | 4.29 | 0.11 | 0.14 | | 0.84 |
| 2289 | C57BL/6 | 10 μg | 0.2 mL | 3.46 | 2.87 | | 2.75 | 0.50 | 0.22 | | 0.05 |
| 2291 | C57BL/6 | 10 μg | 0.2 mL | 6.84 | | 4.63 | 4.38 | 4.16 | | 5.71 | 2.42 |
| MAb | 1 μg/mL | | | 103.81 | | | | 99.28 | | | |
| Cells Alone | | | | 2.67 | | | | 0.06 | | | |

Mean values and percentage gated values of >10% are presented in bold face in Table 5. Using the 10% value as an arbitrary cutoff, two ImmunoMouse animals (2258 and 2055) had peak values on Jun. 15, 2004. An additional 9 ImmunoMouse animals had peak values on Sep. 17, 2004 and one mouse, 2067, had a peak value on Sep. 1, 2004. In contrast, only three of the C57BL/6NHsd mice were responsive: 2328, 2329 and 2331. Note that mice 2245 (ImmunoMouse), 2326 (C57BL/6NHsd) and 2333 (C57BL/6NHsd) are not considered to be positive because of a strong prebleed response. In total, not including the mice with strong prebleed responses, 12 of 24 ImmunoMouse animals (50%) had peak responses on days 35, 113 or 128, compared to 3 of 13 C57BL/6 mice (23.1%), an approximate 2-fold difference.

Based on these results, selected mice were boosted. Seven mice received DNA in the same dose, injection route and injection volume as given earlier. Mammalian cells stably transfected with CD19 (CD19+ cells) were used as an antigen boost in eight mice: four mice received 2×10⁶ CD19+ cells by intravenous injection and four received 5×10⁶ CD19+ cells by intraperitoneal injection. The mice were bled on the day of injection (D-194) and again 14 days later (D-208). Sera from the boosted mice were tested by FACS as above. The selected mice, dose, injection route and FACS results are presented in Tables 6 and 7.

TABLE 6
FACS Analysis of Anti-CD19 Following Boosts with DNA or CD19+ Cells: Mean Fluorescence

| | | | | | | | Mean Values | | | | |
|---|---|---|---|---|---|---|---|---|---|---|---|
| | | Original | Original | | | | | D-112 | D-128 | D-194 | D-208 |
| | | DNA | Inj | D-194 | D-194 | D-194 | | Sep. 01, | Sep. 17, | Nov. 22, | Dec. 06, |
| ID | Strain | Dose | Vol | Boost | Dose | Inj Rt | Prebleed | 2004 | 2004 | 2004 | 2004 |
| 2064 | Immuno | 40 μg | 2 mL | Cells | 2 × 10⁶ | IV | 4.08 | 7.70 | 8.21 | 6.34 | 17.90 |
| 2246 | Immuno | 20 μg | 2 mL | Cells | 2 × 10⁶ | IV | 6.58 | 9.86 | 10.29 | 9.16 | 22.89 |
| 2054 | Immuno | 10 μg | 0.2 mL | Cells | 2 × 10⁶ | IV | 4.09 | 7.06 | 6.49 | 9.25 | 402.3 |
| 2328 | C57BL/6 | 40 μg | 2 mL | Cells | 2 × 10⁶ | IV | 3.63 | 6.86 | 10.58 | 8.81 | 50.25 |
| 2066/2258 | Immuno | 40 μg | 2 mL | Cells | 5 × 10⁶ | IP | 4.52 | 6.06 | 6.25 | 10.91 | 1040.0 |
| 2092/2344 | Immuno | 10 μg | 2 mL | Cells | 5 × 10⁶ | IP | 4.31 | 5.45 | 11.98 | 71.85 | 1517.7 |
| 2056 | Immuno | 40 μg | 2 mL | Cells | 5 × 10⁶ | IP | 4.23 | 4.68 | 4.38 | 5.70 | 2623.3 |
| 2331 | C57BL/6 | 20 μg | 2 mL | Cells | 5 × 10⁶ | IP | 3.73 | 5.15 | 4.35 | 5.08 | 108.11 |
| 2067 | Immuno | 40 μg | 2 mL | DNA | 40 μg | IV | 4.00 | 14.16 | 10.79 | 9.57 | 6.80 |
| 2243 | Immuno | 20 μg | 2 mL | DNA | 20 μg | IV | 4.11 | 7.66 | 7.63 | 11.40 | 10.67 |
| 2091 | Immuno | 10 μg | 2 mL | DNA | 10 μg | IV | 3.65 | 5.14 | 8.80 | 10.34 | 10.09 |
| 2255 | Immuno | 100 μg | 0.1 mL | DNA | 100 μg | IM | 4.16 | 6.01 | 28.30 | 5.86 | 6.47 |
| 2055 | Immuno | 10 μg | 0.2 mL | DNA | 10 μg | IV | 3.79 | 5.40 | 7.29 | 6.72 | 6.13 |
| 2057/2365 | Immuno | 10 μg | 0.2 mL | DNA | 10 μg | IV | 5.70 | 7.79 | 9.15 | 4.91 | 5.69 |
| 2329 | C57BL/6 | 20 μg | 2 mL | DNA | 20 μg | IV | 3.58 | 7.59 | 10.14 | 5.84 | 8.07 |
| MAb | 2 μg/mL | | | | | | 337.23 | | | | |
| No Primary | | | | | | | 3.24 | | | | |

TABLE 7
FACS Analysis of 04-108 Anti-CD19 Following Boosts with DNA or CD19+ Cells:
Percentage Gated

| | | | | | | | Pecentage Gated | | | | |
|---|---|---|---|---|---|---|---|---|---|---|---|
| | | Original | Original | | | | | D-112 | D-128 | D-194 | D-208 |
| | | DNA | Inj | D-194 | D-194 | D-194 | | Sep. 02, | Sep. 17, | Nov. 22, | Dec. 06, |
| ID | Strain | Dose | Vol | Boost | Dose | Inj Rt | Prebleed | 2004 | 2004 | 2004 | 2004 |
| 2064 | Immuno | 40 μg | 2 mL | Cells | 2 × 10⁶ | IV | 0.60 | 2.13 | 2.07 | 2.49 | 65.79 |
| 2246 | Immuno | 20 μg | 2 mL | Cells | 2 × 10⁶ | IV | 3.40 | 12.82 | 12.75 | 11.11 | 86.87 |
| 2054 | Immuno | 10 μg | 0.2 mL | Cells | 2 × 10⁶ | IV | 0.58 | 3.76 | 2.74 | 2.03 | 100.00 |
| 2328 | C57BL/6 | 40 μg | 2 mL | Cells | 2 × 10⁶ | IV | 0.44 | 3.76 | 19.75 | 9.75 | 99.23 |
| 2066/2258 | Immuno | 40 μg | 2 mL | Cells | 5 × 10⁶ | IP | 0.69 | 1.67 | 1.46 | 20.78 | 99.23 |
| 2092/2344 | Immuno | 10 μg | 2 mL | Cells | 5 × 10⁶ | IP | 0.57 | 1.26 | 28.07 | 96.90 | 100.00 |
| 2056 | Immuno | 40 μg | 2 mL | Cells | 5 × 10⁶ | IP | 0.66 | 0.73 | 0.66 | 0.59 | 100.00 |
| 2331 | C57BL/6 | 20 μg | 2 mL | Cells | 5 × 10⁶ | IP | 0.39 | 0.61 | 0.57 | 0.74 | 100.00 |
| 2067 | Immuno | 40 μg | 2 mL | DNA | 40 μg | IV | 0.67 | 39.96 | 17.76 | 5.53 | 2.18 |
| 2243 | Immuno | 20 μg | 2 mL | DNA | 20 μg | IV | 0.58 | 4.93 | 4.59 | 25.32 | 20.18 |
| 2091 | Immuno | 10 μg | 2 mL | DNA | 10 μg | IV | 0.54 | 0.86 | 7.25 | 15.74 | 6.33 |
| 2255 | Immuno | 100 μg | 0.1 mL | DNA | 100 μg | IM | 0.69 | 1.11 | 70.36 | 1.23 | 2.22 |
| 2055 | Immuno | 10 μg | 0.2 mL | DNA | 10 μg | IV | 0.55 | 2.22 | 4.28 | 3.70 | 1.79 |
| 2057/2365 | Immuno | 10 μg | 0.2 mL | DNA | 10 μg | IV | 0.85 | 3.62 | 4.54 | 0.89 | 1.68 |
| 2329 | C57BL/6 | 20 μg | 2 mL | DNA | 20 μg | IV | 0.38 | 5.45 | 6.03 | 1.30 | 8.16 |
| MAb | 2 μg/uL | | | | | | 99.98 | | | | |
| No Primary | | | | | | | 0.24 | | | | |

Of the four mice receiving an IV boost with CD19+ cells, one ImmunoMouse, 2054, had a mean fluorescence of over 400 (D-208 serum), a 40-fold increase over the preboost (D-194) serum. The other mice receiving IV CD19+ cells had increases of between 2.5 and 5.7 fold from D-194 to D-208.

Among the mice injected with CD19+ cells via the intraperitoneal route, the increases were more dramatic. Each of the three ImmunoMouse animals had mean fluorescence values of greater than 1000. The C57BL/6NHsd mouse injected with cells IP also had an increase, but the mean value for this mouse was only 108.

The results indicate that for this experiment, the response of the ImmunoMouse animals was superior to that obtained with the C57BL/6NHsd mice. Subsequent boosting with the antigen (i.e., CD19+ cells) led to a rapid increase in antibody levels, indicating the presence of memory cells induced by genetic immunization.

### Example 7

An OS-9 DNA construct was prepared which contained the OS-9 gene under the control of both a T7 and CMV promoter. For each strain of mice hydrodynamic injections (2.0 mL/mouse) using sterile saline as the carrier (Zhang, et al., Gene Ther. 2004, 11(8):675-82) were used. The total DNA delivered to each mouse was 20 μg per injection. Six ImmunoMouse animals and six BALB/c mice were used per group.

Mice received DNA on experimental days 0, 14, 28 and 42. Serum obtained on D-36 (8 days following the third injection) was tested in a preliminary ELISA on homogenates from 293 cells transfected with the OS-9 gene. The data indicated that several of the mice from both strains responded, an expected result based on the presence of both promoters. The average absorbance value for the ImmunoMouse animals was 0.564, versus 0.394 for the BALB/c mice. While both the ImmunoMouse animals and the BALB/c mice are capable of expressing the OS-9 gene via the CMV promoter, the enhanced results in the ImmunoMouse animals suggest that the presence of the T7 promoter may have further increased expression of the OS-9 construct in the ImmunoMouse. Responding mice of both the ImmunoMouse and the BALB/c type were found to be capable of producing high specificity antibodies upon further immunization with the OS-9 DNA construct. Only a very small number of animals were tested, however, and no statistically significant differences in the response rates to further immunization were detected when comparing the ImmunoMouse and BALB/c animals.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope, and concept of the invention as defined by the appended claims.

What is claimed is:

1. A transgenic mouse whose genome comprises:
a transgene comprising a DNA segment encoding a promoter operably linked to a T7 RNA Polymerase nucleic acid sequence, wherein the transgenic mouse exhibits enhanced responsiveness to genetic immunization as compared to control mice.

2. The transgenic mouse of claim 1, wherein the mouse is a C57BL/6B1 hybrid strain.

3. The transgenic mouse of claim 1, wherein the mouse is heterozygous for the transgene.

4. The transgenic mouse of claim 1, wherein the mouse is homozygous for the transgene.

5. The transgenic mouse of claim 1, wherein the mouse is hemizygous for the transgene.

6. The transgenic mouse of claim 1, wherein the promoter is tissue-specific.

7. The transgenic mouse of claim 1, wherein the promoter is a liver-specific promoter.

8. The transgenic mouse of claim 7, wherein the promoter is ApoA1.

9. The transgenic mouse of claim 1, wherein the promoter is inducible.

10. Progeny of the transgenic mouse of claim 1, wherein the genome of the progeny comprises:
a transgene comprising a DNA segment encoding a promoter operably linked to a T7 RNA Polymerase nucleic acid sequence, wherein the progeny exhibit enhanced receptivity to genetic immunization as compared to control mice.

11. A method of generating antibodies against an immunogen, comprising introducing a polynucleotide construct comprising a nucleic acid sequence encoding an immunogen into a transgenic mouse, wherein the transgenic mouse expresses a T7 RNA Polymerase, wherein the T7 RNA Polymerase interacts with the polynucleotide construct to generate expression of the immunogen, wherein the immunogen elicits an immune response which results in the generation of antibodies.

12. The method of claim 11, wherein the immunogen is an antigen.

13. The method of claim 11, wherein the immunogen comprises an epitope.

14. The method of claim 11, wherein the transgenic mouse further comprises a transgene comprising a DNA segment encoding a promoter operably linked to a T7 RNA Polymerase nucleic acid sequence.

15. The method of claim 14, wherein the promoter is tissue-specific.

16. The method of claim 15, wherein the promoter is a liver-specific promoter.

17. The method of claim 16, wherein the promoter is ApoA1.

18. The method of claim 14, wherein the promoter is inducible.

19. The method of claim 11, wherein the polynucleotide construct further comprises a promoter specifically recognized by the T7 RNA Polymerase.

20. The method of claim 19, wherein the T7 RNA Polymerase binds to the promoter and transcribes the nucleic acid sequence encoding the immunogen.

21. The method of claim 11, further comprising isolating monoclonal antibodies against the immunogen.

22. A method of generating antibodies against a selected immunogen, comprising:
(a) introducing a polynucleotide construct comprising a nucleic acid sequence encoding an immunogen into a transgenic mouse, wherein the genome of the transgenic mouse comprises a transgene comprising a DNA segment encoding a promoter operably linked to a T7 RNA Polymerase nucleic acid sequence;
(b) inducing expression of the T7 RNA Polymerase in the transgenic mouse, wherein the T7 RNA Polymerase interacts with the polynucleotide construct to generate expression of the immunogen;
wherein the immunogen elicits an immune response which results in the generation of antibodies.

23. The method of claim 22, wherein the polynucleotide construct further comprises a promoter specifically recognized by T7 RNA Polymerase.

24. The method of claim 22, wherein the immunogen is an antigen.

25. The method of claim 22, wherein the immunogen comprises an epitope.

26. The method of claim 22, wherein the promoter is tissue-specific.

27. The method of claim 26, wherein the promoter is a liver-specific promoter.

28. The method of claim 27, wherein the promoter is ApoA1.

29. A method of generating cytotoxic T-lymphocytes against an immunogen, comprising introducing a polynucleotide construct comprising a nucleic acid sequence encoding an immunogen into a transgenic mouse, wherein the transgenic mouse expresses a T7 RNA Polymerase, wherein the T7 RNA Polymerase interacts with the polynucleotide construct to generate expression of the immunogen, wherein the inmunogen elicits an immune response which results in the generation of cytotoxic T-lymphocytes.

30. The method of claim 29, wherein the immunogen is an antigen.

31. The method of claim 29, wherein the immunogen comprises an epitope.

32. The method of claim 29, wherein the transgenic mouse further comprises a transgene comprising a DNA segment encoding a promoter operably linked to a T7 RNA Polymerase nucleic acid sequence.

33. The method of claim 32, wherein the promoter is tissue-specific.

34. The method of claim 33, wherein the promoter is a liver-specific promoter.

35. The method of claim 34, wherein the promoter is ApoA1.

36. The method of claim 29, wherein the polynucleotide construct further comprises a promoter specifically recognized by the T7 RNA Polymerase.

37. The method of claim 36, wherein the T7 RNA Polymerase binds to the promoter and transcribes the nucleic acid sequence encoding the immunogen.

38. A transgenic vertebrate whose genome comprises a bacteriophage RNA polymerase as a transgene, wherein said bacteriophage RNA polymerase transgene is capable of being expressed in at least one cell of said transgenic vertebrate.

39. The transgenic vertebrate of claim 38, wherein said vertebrate is a mammal.

40. The transgenic vertebrate of claim 39, wherein said mammal is a mouse.

41. The transgenic vertebrate of claim 38, wherein said bacteriophage RNA polymerase is a T7 RNA polymerase.

42. The transgenic vertebrate of claim 38, wherein said transgene is operably linked to a promoter.

43. A method to produce at least one antibody against an antigen in a transgenic vertebrate whose genome comprises a bacteriophage RNA polymerase transgene, comprising:
a) providing a immunogenic construct comprising the following elements operably linked: i) a promoter sequence cognate to said bacteriophage RNA polymerase, ii) a eukaryotic ribosome recognition sequence, and iii) a sequence encoding said antigen;
b) introducing said immunogenic construct into at least one cell of said transgenic vertebrate; and
c) providing conditions whereby said transgenic vertebrate produces said at least one antibody against said antigen.

44. The method of claim 43, wherein said transgenic vertebrate is a mammal.

45. The method of claim 44, wherein said mammal is a mouse.

46. The method of claim 43, wherein said bacteriophage RNA polymerase is a T7 RNA polymerase.

47. The method of claim 43, further comprising the step of isolating said at least one antibody as a polyclonal antibody.

48. An antibody produced by the method of claim 78.

49. The method of claim 43, further comprising the steps of collecting spleen cells from said transgenic vertebrate, making at least one hybridoma from said spleen cells, and isolating said at least one antibody as a monoclonal antibody from said at least one hybridoma.

50. An antibody produced by the method of claim 49.
